(19) United States
(12) Patent Application Publication
(10) Pub. No.: US 2024/0108287 A1
(43) Pub. Date: Apr. 4, 2024

(54) MEDICAL GUIDEWIRE ASSEMBLY AND/OR ELECTRICAL CONNECTOR

(71) Applicant: Boston Scientific Medical Device Limited, Ballybrit, Galway (IE)

(72) Inventors: John Paul Urbanski, Toronto (CA); Gareth Davies, Toronto (CA)

(21) Appl. No.: 17/769,108

(22) PCT Filed: Oct. 15, 2020
(86) PCT No.: PCT/IB2020/059729

§ 371 Date: Apr. 14, 2022

Related U.S. Application Data
(60) Provisional application No. 62/923,031, filed on Oct. 18, 2019.

(51) Int. Cl. A61B 5/00; A61B 18/14

(52) U.S. Cl. CPC A61B 5/6851; A61B 18/1492; A61B 2018/00351

(57) ABSTRACT

Disclosed is a flexible medical guide wire assembly configured to be inserted into a confined space defined by a living body. A sensor assembly is securely supported by the flexible medical guidewire assembly. This is done in such a way that the sensor assembly and the flexible medical guidewire assembly are movable along the confined space defined by the living body once the flexible medical guidewire assembly is inserted into, and moved along, the confined space defined by the living body. Also disclosed is an electrical-connector assembly having a connector terminal. The connector terminal is configured to be electrically connectable with a terminal portion of a flexible medical guidewire assembly. The terminal portion is electrically connected, via an electrical wire, to a sensor assembly of the flexible medical guidewire assembly.

### TECHNICAL FIELD

This document relates to the technical field of (and is not limited to) a medical device; and (more specifically) this document relates to the technical field of (and is not limited to) a medical guidewire assembly (and/or method therefor); and (even more specifically) this document relates to the technical field of (and is not limited to) an electrical connector for a medical guidewire assembly (and/or method therefor).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with the existing (known) medical devices (also called the existing technology). After much study of, and experimentation with, the existing (known) medical devices, an understanding (at least in part) of the problem and its solution have been identified (at least in part) and are articulated (at least in part) as follows:

Cardiac catheterization is a medical procedure for the insertion of a catheter into a chamber or vessel of the heart (of the patient). This may be done for diagnostic and/or interventional purposes. A common example of cardiac catheterization is coronary catheterization that involves catheterization of the coronary arteries for the treatment of coronary artery disease and myocardial infarctions (heart attacks). Catheterization may be performed in a special laboratory with fluoroscopy and highly maneuverable tables (in which the special laboratory may be equipped with cabinets of catheters, stents, balloons, etc., of various sizes to improve operational efficiency of the special laboratory). Monitors may show (display) the fluoroscopy imaging, electrocardiogram (ECG or EKG) data, images of pressure waves, and more.

Transseptal catheterization is a medical procedure used by interventional cardiologists to gain access to the left atrium of the heart (of a patient). This medical technique was initially introduced for left-sided pressure measurements and has been integrated in a variety of procedures including left atrial ablations and percutaneous mitral valvuloplasties, etc.

Cardiac ablation is a medical procedure to scar or destroy tissue in the heart that is allowing incorrect electrical signals to cause an abnormal heart rhythm. Diagnostic catheters are threaded through blood vessels to the heart where they are used to map the electrical signals of the heart.

Transseptal puncture (TSP) is a medical procedure for gaining access to the left atrium for catheter ablation, hemodynamic assessment of the left heart, left ventricular assist device implantation, percutaneous left atrial appendage closure or mitral valvuloplasty during childhood and adulthood.

Transseptal catheterization procedures may require a number of device exchanges between a known transseptal needle (and any equivalent thereof) (also called a scarring instrument) and a known guidewire. The known transseptal needle may be utilized for access to the transseptal left-heart for medical diagnostic and/or interventional procedures, etc.

A catheter is a flexible medical tube configured to be inserted through a narrow opening into a body cavity (of a patient), such as the bladder (etc.) for removing fluid. Initially, a known guidewire is installed into the patient, and then the catheter is pushed along and guided by the known guidewire (once the catheter is positioned, the guidewire may be removed from the body of the patient). Every device exchange and repositioning of catheters involve uncertainty and/or potentially risky exposure of x-ray radiation to the patient and/or the physician (it may be desirable to keep the exposure of x-ray radiation to a minimum).

A problem with transseptal puncture devices is that they may not be compatible with non-fluoroscopic imaging modalities, and specifically, they may not be optimized to maximize the utility of electroanatomical (EAM) mapping and/or other electrophysiology (EP) Recoding systems (and any equivalent thereof).

Fluoroscopy is an x-ray procedure that makes it possible to see internal organs in motion. Fluoroscopy uses x-rays (which are radioactive) to produce real-time video images. To reduce and/or eliminate the need for fluoroscopy, it may be of value to visualize the tip of a medical device (such as, a tissue-puncture device, an electrode, etc.) on a map (a volume map or a view of the interior of the patient) to be generated by an electroanatomic mapping (EAM) system; this may be performed while also augmenting any imaging provided by an ultrasound system (using tools such as ICE (intracardiac echocardiography)).

It may be of value to obtain information about the spatial position of the medical device (such as the tissue-puncture device, transseptal needle, etc.) mounted to the distal position of a guidewire; it will be appreciated that some physicians may require the usage of fluoroscopy, at times, to ensure that a starter guidewire has tracked safely from the inferior vena cava (IVC) leading into the heart to the superior vena cava (SVC) of the heart (of the patient). Additionally, after removing the transseptal needle, it may be necessary to confirm the path of the guidewire on the left-side of the heart (due to uncertainty associated with the distal position of the guidewire).

It may be valuable to provide a guidewire configured to function as a tissue-puncture device while being optimized for utilization with a non-fluoroscopic imaging mode for the exchange steps (such as, for the exchange of medical devices on the known guidewire) associated with the medical procedure.

The following are some identified problems: known diagnostic catheters (including, for instance, needles or puncture devices within a known catheter) may (A) feature a hub that may not be usable for catheter exchange (medical device exchange); (B) not have sufficient stiffness as utilized, for instance, in a procedure for making a transseptal puncture for cardiac cases, etc.; and/or (C) be utilized for low voltage applications (whereas, a potential solution, in some instance, may be to use a relatively higher voltage delivery device to function, for instance, as an electrosurgical device, etc.).

It may be beneficial to provide a flexible medical guidewire configured for utilization in a minimally invasive medical procedure; a physician may need to deploy additional diagnostic catheters (over the medical guidewire) to perform a desired medical task, such as an electrophysiology study (EPS) as part of a medical therapy. It will be appreciated that sometimes the wire may be used to deliver the diagnostic catheter, and sometimes the wire may be used to deliver the sheath that may ultimately direct the diagnostic catheter.

It may be beneficial to provide a medical guidewire configured to sense a signal (such as an electrical signal, a magnetic signal) to be input to a signal-recording system, for subsequent signal analysis. The signal (preferably, a relatively high-precision signal) may be associated with an electrocardiogram (ECG) in any configuration, such as a unipolar configuration or a bipolar configuration), etc.

It may be beneficial to provide a medical guidewire having at least one or more sensor devices (such as, a plurality of electrodes, etc.) that is/are supported by the medical guidewire. The sensor device may improve spatial resolution, simplify workflows and/or provide material savings. For instance, after catheterization of the heart chambers is completed with the catheterization device that is mounted to the tip of the medical guidewire, the medical guidewire may then be repositioned (or parked) in other regions of the heart to facilitate the recording of signals (provided as sensor output signals from a sensor mounted to the medical guidewire) during therapy. Specific regions of the heart that may require signal recordings (such as, during electrophysiology studies) may include the right atrium (RA), the right ventricle (RV) and/or the coronary sinus (CS), amongst others. It may be also beneficial to provide a medical guidewire configured to emit (convey or transmit) a signal in predetermined manner, such as a bipolar manner and/or a unipolar manner (preferably in addition the medical guidewire being configured to receive a signal). Diagnostic electrophysiology (EP) catheters (also called EP diagnostic catheters) may be used for temporary intra-cardiac sensing, recording, stimulation, and mapping. EP diagnostic catheters may be indicated for both recording and pacing of heart tissue (as or when needed). Additionally, known Electroanatomic mapping (EAM) systems may require emission of a sensing signal from an emitter device (a signal source), and may require the signal to be sensed by another device (a signal receiving device), such as a receiver pad placed on a patient, etc.

It may be beneficial to utilize at least one embodiment in research and development projects and/or medical clinical environments.

It may be beneficial to provide a medical guidewire that adds at least one medical function that may be performed by multiple separate medical devices.

To mitigate, at least in part, at least one problem associated with the existing technology, there is provided (in accordance with a major aspect) an apparatus. The apparatus includes and is not limited to (comprises) a synergistic combination of a flexible medical guidewire assembly and a sensor assembly. The flexible medical guidewire assembly is configured to be inserted into a confined space defined by a living body. The sensor assembly is securely supported by the flexible medical guidewire assembly. This is done in such a way that the sensor assembly and the flexible medical guidewire assembly are movable along the confined space defined by the living body once the flexible medical guidewire assembly is inserted into, and moved along, the confined space defined by the living body. It will be appreciated that the detailed description provides a description of embodiments for the flexible medical guidewire assembly.

To mitigate, at least in part, at least one problem associated with the existing technology, there is provided (in accordance with a major aspect) a method. The method includes and is not limited to (comprises) the following steps (operations): an operation (A) providing the flexible medical guidewire assembly configured to be inserted into a confined space defined by a living body, and an operation (B) providing a sensor assembly securely supported by the flexible medical guidewire assembly in such a way that the sensor assembly and the flexible medical guidewire assembly are movable along the confined space defined by the living body once the flexible medical guidewire assembly is inserted into, and moved along, the confined space defined by the living body. It will be appreciated that the detailed description provides a description of embodiments for the flexible medical guidewire assembly. Preferably, the method if for utilizing a flexible medical guidewire assembly, comprising: (A) providing the flexible medical guidewire assembly configured to be inserted into a confined space defined by a living body, in which there is a sensor assembly securely supported by the flexible medical guidewire assembly; and (B) inserting, at least in part, the sensor assembly and the flexible medical guidewire assembly into the confined space defined by the living body; and (C) moving, at least in part, the sensor assembly and the flexible medical guidewire assembly along the confined space defined by the living body once the flexible medical guidewire assembly is inserted into the confined space defined by the living body.

To mitigate, at least in part, at least one problem associated with the existing technology, there is provided (in accordance with a major aspect) an apparatus. The apparatus includes and is not limited to (comprises) an electrical-connector assembly having a connector terminal. The connector terminal is configured to be electrically connectable with a terminal portion (a wire terminal) of a flexible medical guidewire assembly. The flexible medical guidewire assembly is configured to be inserted into a confined space defined by a living body. The terminal portion is electrically connected, via an electrical wire, to a sensor assembly of the flexible medical guidewire assembly. It will be appreciated that the detailed description provides a description of embodiments for the electrical-connector assembly.

To mitigate, at least in part, at least one problem associated with the existing technology, there is provided (in accordance with a major aspect) a method. The method includes and is not limited to (comprises) providing an electrical-connector assembly having a connector terminal. The connector terminal is configured to be electrically connectable with a terminal portion of a flexible medical guidewire assembly. The flexible medical guidewire assembly is configured to be inserted into a confined space defined by a living body. The terminal portion is electrically connected, via an electrical wire, to a sensor assembly of the flexible medical guidewire assembly. It will be appreciated that the detailed description provides a description of embodiments for the electrical-connector assembly. Preferably, the method is for utilizing an electrical-connector assembly, comprising: (A) providing the electrical-connector assembly having a connector terminal being configured to be electrically connectable with a terminal portion of a flexible medical guidewire assembly, in which the flexible medical guidewire assembly is configured to be inserted into a confined space defined by a living body, and in which the terminal portion is electrically connected, via an electrical wire, to a sensor assembly of the flexible medical guidewire assembly; and (B) electrically connecting the connector terminal of the electrical-connector assembly to the terminal portion of the flexible medical guidewire assembly.

To mitigate, at least in part, at least one problem associated with the existing technology, there is provided (in accordance with a major aspect) a method. The method includes and is not limited to (comprises) the following steps (operations): operation (A), operation (B), operation (C), and operation (D). Operation (A) includes utilizing a medical-imaging system (such as, an intracardiac echocardiography (ICE) system, an electroanatomic mapping (EAM) system, etc., and any equivalent thereof) to generate (register, display, etc.) a medical image (such as, a voltage map, a geometry-capturing system configured for capturing tissue geometry, etc., and any equivalent thereof) of the relevant anatomy of the patient (such as, the right atrium of the heart, the septum that separates the right and left atria of the heart, etc.). Operation (B) includes inserting (deploying, advancing, moving, etc.), once or after the medical image is generated by the medical-imaging system and is displayed to a doctor performing the procedure), a flexible medical guidewire assembly toward the relevant anatomy of the patient (that is, inserting the flexible medical guidewire assembly along the confined space defined by the living body and toward the relevant anatomy of the patient). Operation (C) includes utilizing the medical-imaging system to detect (while the flexible medical guidewire assembly is inserted into the patient toward the relevant anatomy of the patient) the spatial position of a sensor assembly (that is fixedly mounted to a portion of the flexible medical guidewire assembly) so that the spatial position of the portion (such as the tip) of the flexible medical guidewire assembly may be identified (detected) by the medical-imaging system (that is, the position of the portion of the flexible medical guidewire assembly may be displayed to the doctor while the flexible medical guidewire assembly is inserted into the patient toward the relevant anatomy of the patient). Operation (D) includes guiding (once the doctor has made a determination that the portion of the flexible medical guidewire assembly has reached, or has been placed proximate to, the relevant anatomy of the patient, and the flexible medical guidewire assembly is held spatially motionless relative to the relevant anatomy of the patient), the insertion of a medical instrument (along a length of the flexible medical guidewire assembly) toward the relevant anatomy of the patient (that is, the medical instrument is moved along the confined space defined by the living body toward the relevant anatomy of the patient since the flexible medical guidewire assembly has reached a position located proximate to the relevant anatomy of the patient), and this manner the medical instrument may be activated (or utilized) for medical treatment of the relevant anatomy of the patient. It will be appreciated that other operations may include reversing the operation steps described above for deactivation and/or retraction of the medical instrument from the patient, and retraction of the flexible medical guidewire assembly from the patient, etc. It will be appreciated that for the case where the flexible medical guidewire assembly includes a heating device, and additional operation may include activating the heating device (once the doctor has made a determination (based on the generated medical image provided by the medical-imaging system) that the portion of the flexible medical guidewire assembly has reached, or has been placed proximate to, the relevant anatomy of the patient, and the flexible medical guidewire assembly is held spatially motionless relative to the relevant anatomy of the patient). It will be appreciated that in view of the detailed description, further operational steps may be added.

To mitigate, at least in part, at least one problem associated with the existing technology, there is provided (in accordance with a major aspect) a method. The method includes and is not limited to (comprises) the following steps (operations): operation (A), operation (B), operation (C), and operation (D). Operation (A) includes utilizing a medical-imaging system (such as, an intracardiac echocardiography (ICE) system, an electroanatomic mapping (EAM) system, etc., and any equivalent thereof) to generate (register, display, etc.) a medical image (such as, a volume map, a geometry-capturing system configured to capture tissue geometry, etc., and any equivalent thereof) of the relevant anatomy of the patient (such as, the right atrium of the heart, the septum that separates the right and left atria of the heart, etc.). Operation (B) includes inserting (deploying, advancing, moving, etc.), once or after the medical image is generated by the medical-imaging system and is displayed to a doctor performing the procedure, etc.), a flexible medical guidewire assembly toward the relevant anatomy of the patient (that is, inserting the flexible medical guidewire assembly along the confined space defined by the living body and toward the relevant anatomy of the patient). Operation (C) includes utilizing the medical-imaging system to detect (while the flexible medical guidewire assembly is inserted into the patient toward the relevant anatomy of the patient) the spatial position of a sensor assembly (that is fixedly mounted to a portion of the flexible medical guidewire assembly) so that the spatial position of the portion (such as the tip) of the flexible medical guidewire assembly may be identified (detected) by the medical-imaging system (that is, the position of the portion of the flexible medical guidewire assembly may be displayed to the doctor while the flexible medical guidewire assembly is inserted into the patient toward the relevant anatomy of the patient). Operation (D) activating a heating device (located and fixedly positioned proximate to the portion of the flexible medical guidewire assembly) once the doctor has made a determination that the portion of the flexible medical guidewire assembly has reached, or has been placed proximate to, the relevant anatomy of the patient, and the flexible medical guidewire assembly is held spatially motionless relative to the relevant anatomy of the patient. It will be appreciated that the medical instrument, for this method, may be deployed or may not be deployed in conjunction with deployment of the heating device, in that the deployment of the medical instrument is optional for this method. It will be appreciated that other operations may include reversing the operation steps described above for deactivation and/or retraction of the medical instrument from the patient (if the medical instrument is utilized), and/or retraction of the flexible medical guidewire assembly from the patient, etc. It will be appreciated that in view of the detailed description, further operational steps may be added.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1A, FIG. 1B, FIG. 1C and FIG. 1D depict side perspective views of embodiments of a flexible medical guidewire assembly; and

FIG. 2A and FIG. 2B depict front views of embodiments of the flexible medical guidewire assembly of FIG. 1A, FIG. 1B, FIG. 1C and/or FIG. 1D; and

FIG. 3 depicts a front perspective view of an embodiment of the flexible medical guidewire assembly of FIG. 1D;

FIG. 4 depicts a front perspective view of an embodiment of the flexible medical guidewire assembly of FIG. 1D; and

FIG. 5 depicts a front perspective view of an embodiment of the flexible medical guidewire assembly of FIG. 1D; and

FIG. 6A and FIG. 6B depict an axial cross-sectional view (FIG. 6A) and a radial cross-sectional view (FIG. 6B) of embodiments of the flexible medical guidewire assembly of FIG. 1D; and

FIG. 7A, FIG. 7B, FIG. 7C, FIG. 7D and FIG. 7E depict radial cross-sectional views of embodiments of the flexible medical guidewire assembly of FIG. 1D; and

FIG. 8A and FIG. 8B depict radial cross-sectional views of embodiments of the flexible medical guidewire assembly of FIG. 1D; and

FIG. 9A depicts a side view of an embodiment of the flexible medical guidewire assembly of FIG. 1D; and

FIG. 9B, FIG. 9C, FIG. 9D, FIG. 9E and FIG. 9F depict side views of embodiments of an electrical connector configured to be connectable to the flexible medical guidewire assembly of FIG. 9A; and

FIG. 10A and FIG. 10B depict side views of embodiments of an electrical connector configured to be connectable to the flexible medical guidewire assembly of FIG. 1D; and

FIG. 11A and FIG. 11B depict perspective views of embodiments of the flexible medical guidewire assembly of FIG. 1D; and

FIG. 11C depicts a side view of an embodiment of an electrical connector configured to be connectable to the flexible medical guidewire assembly of FIG. 11A and/or FIG. 11B; and

FIG. 12A depicts a perspective view of an embodiment of the flexible medical guidewire assembly of FIG. 1D; and

FIG. 12B and FIG. 12C depict side views of embodiments of an electrical connector configured to be connectable to the flexible medical guidewire assembly of FIG. 12A.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

flexible medical guidewire assembly 102
sensor assembly 104
core element 106
core terminal portion 106A
core insulation layer 106B
jacket element 108
jacket portal 108A
first jacket channel 109A
second jacket channel 109B
nth jacket channel 109N
tip portion 110
heating device 112
heating wire 113
magnetic sensor device 402
electrical sensor device 404
first electrical sensor device 404A
nth electrical sensor device 404N
electrical wire 405
first electrical wire 405A
second electrical wire 405B
nth electrical wire 405N
braided element 406
first wire insulation layer 407A
second wire insulation layer 407B
nth wire insulation layer 407N
terminal portion 409
first terminal portion 409A
second terminal portion 409B
nth terminal portion 409N
electrical-connector assembly 810
connector terminal 811
first connector terminal 811A
second connector terminal 811B
third connector terminal 811D
nth connector terminal 811N
connector wire 812
first connector wire 812A
second connector wire 812B
fourth connector wire 812D
nth connector wire 812N
handle 814
housing assembly 816
spring member 818
push button 820
conductor 822
connector channel 824
complementary profile 826
first pole 828A
second pole 828B
wire connections 832
spring member 833
leaf spring 836
mating slot 838
connector terminals 840
medical instrument 900
living body 902
signal measurement system 904
sensor-interface system 906
grounding element 908
flat radial end face 911

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the claim is defined by the claims (in which the claims may be amended during patent examination after the filing of this application). For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the invention is limited to the subject matter provided by the claims, and that the invention is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

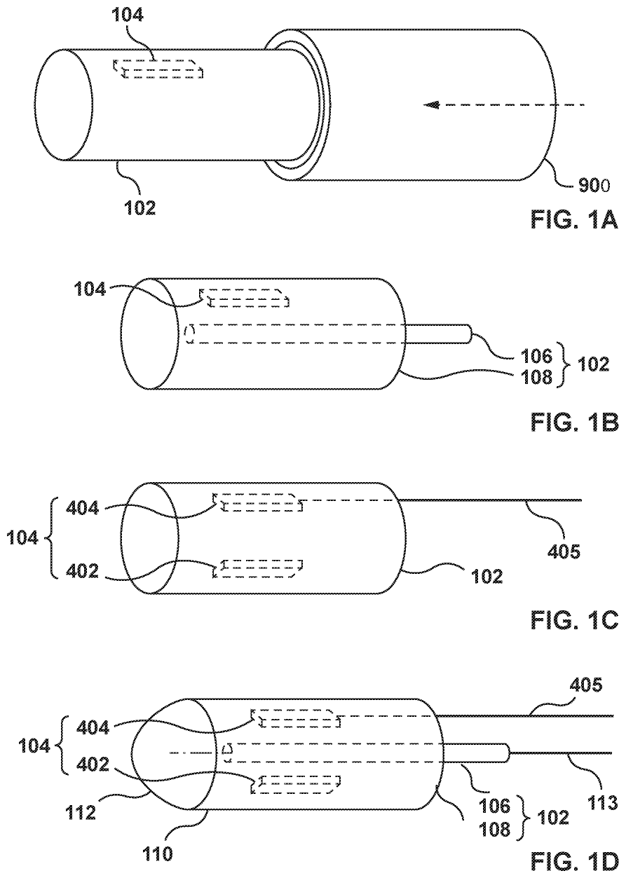

FIG. 1A, FIG. 1B, FIG. 1C and FIG. 1D depict side perspective views of embodiments of a flexible medical guidewire assembly 102.

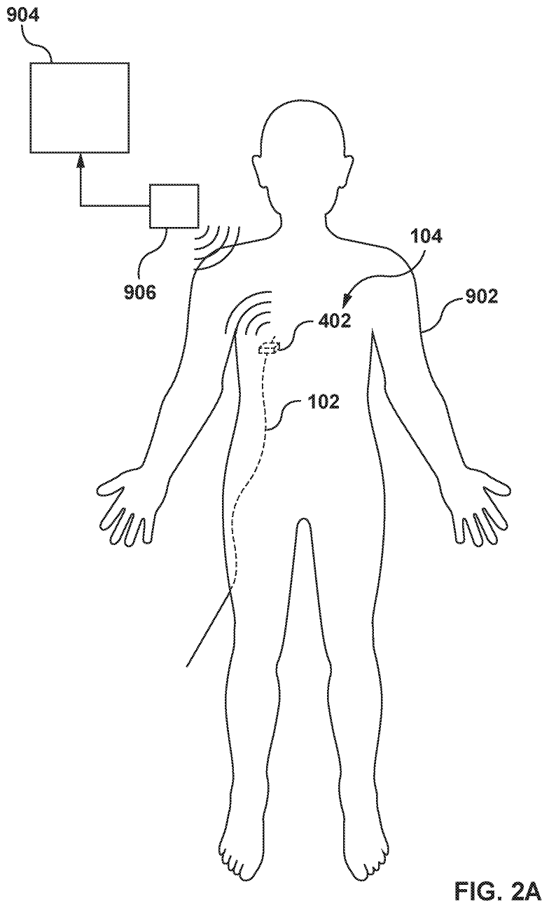

Referring to the embodiment as depicted in FIG. 1A, there is depicted an apparatus including and not limited to (comprising) a synergistic combination of a flexible medical guidewire assembly 102 and a sensor assembly 104. The flexible medical guidewire assembly 102 is configured to be inserted into a confined space defined by a living body 902. An embodiment of the living body 902 is depicted in FIG. 2A or FIG. 2B. The living body 902 may include a human body, etc. The sensor assembly 104 is securely supported by (is configured to be supported by) the flexible medical guidewire assembly 102. This is done in such a way that the sensor assembly 104 and the flexible medical guidewire assembly 102 are movable along the confined space defined by the living body 902 once the flexible medical guidewire assembly 102 is inserted into, and moved along, the confined space defined by the living body 902. The flexible medical guidewire assembly 102 may include any type of a flexible material. The sensor assembly 104 may include any type of sensor assembly.

Referring to the embodiment as depicted in FIG. 1A, the flexible medical guidewire assembly 102 (preferably) includes a sensor assembly 104 configured to respond to a stimulus (such as heat, light, sound, pressure, magnetism, or a particular motion and any equivalent thereof) and transmit a resulting signal (such as an impulse for signal measurement or for operating a control function, etc.). Preferably, the flexible medical guidewire assembly 102 is configured to include (support) a sensor assembly 104. The sensor assembly 104 may include a radiation emitter, an energy emitter, an energy receiver, a magnetic flux emitter, a rare earth magnet, etc., and any equivalent thereof. In accordance with an embodiment, the flexible medical guidewire assembly 102 and the sensor assembly 104 are configured to be selectively attachable to, and selectively detachable from, each other.

Referring to the embodiment as depicted in FIG. 1A, the flexible medical guidewire assembly 102 is (preferably) configured to guide the insertion of a medical instrument 900 (such as, a catheter, etc. and any equivalent thereof) into the confined space defined by the living body 902 (as depicted in FIG. 2A or FIG. 2B) once the flexible medical guidewire assembly 102 is inserted into the confined space defined by the living body 902. The flexible medical guidewire assembly 102 is (preferably) configured to facilitate catheter exchange (the exchange of medical devices or the removal and insertion of medical devices). The flexible medical guidewire assembly 102 includes (preferably) a relatively thin and flexible wire (an elongated flexible shaft) configured to be inserted into a confined or tortuous space (such as the confined space defined by the living body 902). The flexible medical guidewire assembly 102 provides (preferably) a guide for subsequent insertion of the medical instrument 900. The medical instrument 900 may include a relatively stiffer and/or bulkier medical device (medical instrument), such as a catheter (medical catheter), etc. The medical device has a stiffness that is relatively stiffer compared to the stiffness of the flexible medical guidewire assembly 102. The catheter provides (includes) a flexible tube (made from a medical grade material) configured to be inserted through a narrow opening into a body cavity space (the confined space defined by the living body 902), such as the bladder, for removing a fluid therefrom. The catheter may be configured to be inserted into the body to treat diseases or perform a surgical procedure. By modifying the material or adjusting the way catheters are manufactured, it is possible to tailor catheters for cardiovascular, urological, gastrointestinal, neurovascular, and ophthalmic applications. The catheter may be configured to allow drainage, the administration of fluids or gases, access by surgical instruments, and also the performance of a wide variety of other tasks. The process of inserting a catheter is catheterization. The catheter may include a thin and flexible tube (a soft catheter), and catheters may be available in varying levels of stiffness depending on the medical task or application. It will be appreciated that for the case where the catheter is too soft, the flexible medical guidewire assembly 102 may be initially inserted into the (same) body cavity, and then the catheter is inserted into the body cavity by having the flexible medical guidewire assembly 102 guide the catheter as the catheter is pushed into the body cavity.

Referring to the embodiment as depicted in FIG. 1A, the flexible medical guidewire assembly 102 is (preferably) configured for insertion into the confined space defined by the living body 902 in a manner that is assisted only by the user (the doctor or the technician) or by any medical device previously inserted and positioned in the confined space defined by the living body 902, etc. The flexible medical guidewire assembly 102 is (preferably) impermeable by a bodily fluid located in the confined space defined by the living body 902 (once the flexible medical guidewire assembly 102 is inserted into the confined space defined by the living body 902). In accordance with a preferred embodiment, the flexible medical guidewire assembly 102 has (preferably) an outer diameter of about two (2) millimeters (mm) and an elongated length of about 30 inches to about 90 inches; it will be appreciated that other dimensions of the flexible medical guidewire assembly 102 are possible. In accordance with an embodiment, the flexible medical guidewire assembly 102 has (preferably) an elongated length of about 150 centimeters (cm) to about 260 cm, and an outer diameter of about 0.025 inches to about 0.035 inches.

Referring to the embodiment as depicted in FIG. 1B, the flexible medical guidewire assembly 102 includes (preferably) a synergistic combination of a core element 106 and a jacket element 108 (also called a jacket portion, an envelope, an outer coating, etc.) surrounding the core element 106 (also called a mandrel). The core element 106 and the jacket element 108 extend along an elongated length of the flexible medical guidewire assembly 102. The flexible medical guidewire assembly 102 (preferably) has a circular cross-sectional section or profile (it will be appreciated that other profile shapes may be utilized).

Referring to the embodiment as depicted in FIG. 1B, the core element 106 includes (preferably) a stiff internal mandrel. The core element 106 provides (preferably) additional stiffness to the flexible medical guidewire assembly 102.

Referring to the embodiment as depicted in FIG. 1B, the core element 106 includes (preferably), in accordance with an option, SAE (Society of Automotive Engineering) Type 304 Stainless Steel. SAE Type 304 stainless steel contains both chromium (from between 15% to 20%) and nickel (between 2% to 10.5%) metals as the main non-iron constituents. The core element 106 includes (in accordance with another option) superelastic nitinol. Nitinol alloys exhibit two closely related and unique properties: shape memory effect (SME) and superelasticity (SE; also called pseudoelasticity or PE). Shape memory is the ability of nitinol to undergo deformation at one temperature, then recover its original, undeformed shape upon heating above its transformation temperature.

Superelasticity occurs at a narrow temperature range just above its transformation temperature; in this case, no heating is necessary to cause the undeformed shape to recover, and the material exhibits enormous elasticity, from about ten (10) to thirty (30) times that of ordinary metal.

Referring to the embodiment as depicted in FIG. 1B, the core element 106 provides (preferably) a combination of electrical and mechanical properties. It will be appreciated that it may not be necessary for the core element 106 to be electrically connected or to act as an electrical line (electrical wire).

Referring to the embodiment as depicted in FIG. 1B, the core element 106 has (preferably) a degree of stiffness that is constant (provides constant stiffness) along a length of the flexible medical guidewire assembly 102, or the degree of stiffness may vary (a variable stiffness) along a length of the flexible medical guidewire assembly 102.

Referring to the embodiment as depicted in FIG. 1B, the core element 106 may include a hollow tube, such as a hypotube stiffening member. A hypotube is a long metal tube with micro-engineered features along a length of the tube. In accordance with an optional embodiment, the hollow tube is configured to receive and house an electrical wire 405. The hollow tube may provide a modular configuration, and the hollow tube may provide a cut-out to control (adjust) the flexibility (stiffness) of the hollow tube, etc.

Referring to the embodiment as depicted in FIG. 1C, the flexible medical guidewire assembly 102 (preferably) includes the sensor assembly 104. The sensor assembly 104 (preferably) includes a magnetic sensor device 402. The magnetic sensor device 402 may include a rare-earth magnet, a permanent magnet, and/or an electro-magnet. The magnetic sensor device 402 includes a material configured to exhibit at least one property of magnetism, such as attracting other iron-containing objects or aligning itself in an external magnetic field, etc.

Referring to the embodiment as depicted in FIG. 1C, the sensor assembly 104 (preferably) includes an electrical sensor device 404 (a biosensor, etc.) configured to transmit a signal, in which the signal may be transmitted via an electrical wire 405 and/or via a radio transmitter device (known and not depicted). The electrical sensor device 404 is configured to transmit (emit) an electrical signal (a biosignal) and/or receive an electrical signal (a biosignal). The electrical sensor device 404 is configured to detect an event and/or a change in its environment, and send (transmit) the information to other electronics (such as a computer processor, etc.). A biosensor is an analytical device configured to detect a chemical substance, and may combine a biological component with a physicochemical detector. A biosignal is a signal in a living being (body) that may be continually measured and monitored, and may refer to a bioelectrical signal, and may refer to both electrical and non-electrical signals (both of which may be time-varying signals). It will be appreciated that, in accordance with a preferred embodiment, the sensor assembly 104 may include a synergistic combination of the electrical sensor device 404 and the magnetic sensor device 402.

Referring to the embodiment as depicted in FIG. 1C, the flexible medical guidewire assembly 102 (preferably) includes an electrical wire 405 extending along (a length of) the flexible medical guidewire assembly 102. The electrical wire 405 is electrically connected (coupled either directly or indirectly) to the sensor assembly 104. The electrical wire 405 extends from the sensor assembly 104 toward a terminal end of the flexible medical guidewire assembly 102 (such as, a proximal end of the flexible medical guidewire assembly 102), and terminates (at a terminal point or a terminal contact) at the terminal end of the flexible medical guidewire assembly 102. The electrical wire 405 may be called an electrical line. For the case where the sensor assembly 104 includes a plurality of sensors, a plurality of the electrical wires 405 are deployed (once per deployed or installed sensor assembly). The electrical wire 405 may include a miniaturized electrical wire (such as from about 34 to about 44 AWG (American Wire Gauge)) to free up cross-sectional space located within the flexible medical guidewire assembly 102. The electrical wire 405 may include copper, stainless steel, nitinol, etc. The electrical wire 405 may include a flat ribbon wire having a rectangular cross-section, with a thickness (for instance, from about 0.002 inches or less), and preferably minimizes the impact on overall wire outer diameter, etc. The electrical wire 405 may have a total end-to-end DC resistance that is minimized. The electrical wire 405 may have a total end-to-end DC resistance (for instance, from about 20 ohms or less).

Referring to the embodiment as depicted in FIG. 1C, the flexible medical guidewire assembly 102 (preferably) is adapted such that the electrical wire 405 is not included, and the sensor assembly 104 includes a radio transmitter (known and not depicted) configured to transmit a radio signal. The radio transmitter is positioned on the sensor assembly 104 (the radio transmitter is an option for not using the electrical wire 405). It will be appreciated that the radio transmitter is an equivalent to the electrical wire 405.

Referring to the embodiment as depicted in FIG. 1D, the flexible medical guidewire assembly 102 (preferably) includes a synergistic combination of a core element 106 and a jacket element 108. The core element 106 (also called a mandrel) is electrically conductive. The jacket element 108 is electrically insulative and surrounds the core element 106.

Referring to the embodiment as depicted in FIG. 1D, a tip portion 110 is positioned at an end section of the core element 106 and the jacket element 108. A heating device 112 is mounted to the tip portion 110 of the flexible medical guidewire assembly 102. The heating device 112 is electrically connected to the core element 106 (which, for this embodiment, is electrically conductive). The heating wire 113 is electrically connectable to (and disconnectable from) the proximal end (user-accessible end) of the core element 106. In accordance with a preferred embodiment, the heating device 112 includes (and is not limited to) an RF (radio frequency) emitter. The RF emitter is configured to provide (emit) heat energy of a sufficient amount to remove, cauterize and/or puncture (preferably by a process of cauterization) adjacently positioned tissue of the body (of a patient) (the tissue is positioned adjacently (proximate) to the heating device 112 once the flexible medical guidewire assembly 102 is inserted into the confined space defined by the living body 902, and once the heating device 112 is activated accordingly). Cauterization includes management (application and/or removal) of thermal energy proximate to living tissue for the purpose of forming a void, groove and/or a passageway through the living tissue while sealing off blood vessels in the living tissue and preventing unwanted bleeding from the living tissue (thereby promote easier healing). Preferably, a proximal end of the core element 106 is configured to be electrically connectable to a heating wire 113 (in which the heating wire 113 is configured to provide electricity to the heating device 112 via the core element 106). In accordance with a preferred embodiment, there is provided the flexible medical guidewire assembly 102 with at least one sensor assembly 104 in combination with the radio frequency emitter (which is a tissue-puncture device); the radio frequency emitter is configured to emit thermal energy of sufficient quantity to cauterize tissue (tissue wall) positioned proximate to the radio frequency emitter (thereby forming a hole or passageway through the tissue or tissue wall); this provides a technical solution for using less fluoroscopy techniques and systems during a medical procedure and/or treatment (this arrangement may reduce or eliminate the need for a switch between sensing and energy delivery). In accordance with an embodiment, the heating device 112 is further configured to receive and/or record electrical signals, and/or is configured for electrosurgical purposes.

Referring to the embodiment as depicted in FIG. 1D, the jacket element 108 may be called an outer layer or an insulation layer (an electrically insulative material or electrical insulation material). The jacket element 108 has, houses or contains the electrical wire 405. For instance, the jacket element 108 may include PTFE (an extrusion of PTFE), and any equivalent thereof. Polytetrafluoroethylene (PTFE) is a synthetic fluoropolymer of tetrafluoroethylene. The jacket element 108 may include (define) at least one or more lumens (elongated voids) configured to receive the electrical wire 405, etc.

Referring to the embodiment as depicted in FIG. 1D, the heating device 112 (preferably) includes an electrode (also called a distal electrode, a radio frequency (RF) electrode, etc.). The electrode may include stainless steel, nitinol, platinum and iridium alloy (blend), or a mix of thereof. The electrode may form a geometric shape of a semi-spherical dome, having (preferably) a diameter (for instance, from about 0.015 inches to about 0.032 inches, and more preferably a diameter about 0.024 inches). An exposed metallic (conductive) area is, preferably, sized from about 1.2 millimeters (mm\hat{ }2) to about 2.4 mm\hat{ }2 to ensure a relatively higher current density for the case where from about 270 Vrms (volts root mean square) to about 400 Vrms is delivered in a unipolar manner (i.e., to a grounded patient) to achieve tissue puncture (RF puncture) of adjacently positioned tissue of the patient (once the flexible medical guidewire assembly 102 is inserted into the confined space defined by a living body 902, and once the heating device 112 is activated). The electrode may be configured to provide a blunt surface such that the electrode does not mechanically puncture the tissue inadvertently. The electrode, preferably, becomes effectively sharp (for removing tissue) once RF energy is applied to the electrode and transmitted to the tissue.

Referring to the embodiments as depicted in FIG. 1A, FIG. 1B, FIG. 1C and FIG. 1D, the following describes additional technical features for the embodiments of the flexible medical guidewire assembly 102. It will be appreciated that these are preferred embodiments and are not essential for the flexible medical guidewire assembly 102. Preferably, the flexible medical guidewire assembly 102 is configured for medical device exchange (such as catheter exchange). Preferably, a working length of the flexible medical guidewire assembly 102 may be sufficient for medical device exchange (guidewire lengths may be twice as long as the medical devices being exchanged by utilizing the flexible medical guidewire assembly 102). Preferably, the flexible medical guidewire assembly 102 is configured for transseptal exchange. Preferably, the flexible medical guidewire assembly 102 has a flexural rigidity (preferably from about 0.001 Nm2 (Newton per Square Metre Pressure Unit) to about 0.002 Nm2 along the majority of the length of the flexible medical guidewire assembly 102). Preferably, the flexible medical guidewire assembly 102 has a stiffness similar (equivalent) to a Type 304 spring tempered stainless steel wire (for instance of about 0.018 inches in diameter) in the region of the flexible medical guidewire assembly 102 that is positioned across the atrial septum (of the heart). This preference may depend on many anatomical and environmental conditions; it will be appreciated that some procedures may benefit from more stiffness, some less. Preferably, the flexible medical guidewire assembly 102 may include a core element 106 (also called a stiff core mandrel) with a jacket element 108 (also called a flexible jacket layer) placed or positioned overtop (of the core element 106) to improve shape retention (of the flexible medical guidewire assembly 102), and/or to provide a smooth overall elongated profile (of the flexible medical guidewire assembly 102). In accordance with an embodiment, the core element 106 has an outer diameter range (for instance of about 0.015 inches to about 0.030 inches). In accordance with an embodiment, the core element 106 includes stainless steel and/or nitinol. In accordance with an embodiment, the jacket element 108 includes a material that provides a minimal contribution to stiffness of the flexible medical guidewire assembly 102. In accordance with an embodiment, the jacket element 108 includes an electrical insulation material (such as PTFE (polytetrafluoroethylene)). In accordance with an embodiment, the jacket element 108 includes a flexible steel coil. In accordance with an embodiment, the jacket element 108 has a thickness such that the outer diameter of the flexible medical guidewire assembly 102 ranges (preferably) from about 0.032 inches to about 0.035 inches.

In accordance with an embodiment, the distal end of the flexible medical guidewire assembly 102 is curved and flexible to protect the tissue (of the patient) during advancement of the flexible medical guidewire assembly 102 through vessels (of the patient). In accordance with an embodiment, the flexible medical guidewire assembly 102 includes radiovisible materials. In accordance with an embodiment, the flexible medical guidewire assembly 102 is configured to withstand handling (such as user forces and/or environmental forces, etc.) to be experienced during a medical (cardiac) procedure (treatment or diagnostic procedure). In accordance with an embodiment, the flexible medical guidewire assembly 102 complies with international medical standards mandated for minimum tensile forces, such as about ten (10) N (Newton), for medical guidewires dimensioned in the range from about 0.032 inches to about 0.035 inches (preferably without any loosening or separation of the sections or portions of the flexible medical guidewire assembly 102). In accordance with an embodiment, the electrical connections of the flexible medical guidewire assembly 102 (at the distal end) require sufficient electrical insulation end-to-end to mitigate interference(s) (environmental interference). In accordance with an embodiment, the flexible medical guidewire assembly 102 complies with international medical standards for electrosurgical devices that mandate the minimum electrical insulation performance of the guidewires (for the protection of the patient and/or the medical technician). In accordance with an embodiment, the flexible medical guidewire assembly 102 includes a radio frequency (RF) device (also called a heating device) configured to deliver (preferably) from about 270 Vrms to 400 Vrms (Volts root mean square) (preferably in a unipolar configuration) for distal tissue puncture or tissue removal. In accordance with an embodiment, the flexible medical guidewire assembly 102 includes the jacket element 108; the jacket element 108 includes an insulation material having a thickness of about 0.00275 inches (such as, PTFE) or more, in which the jacket element 108 is positioned over any voltage carrying conductors positioned in the flexible medical guidewire assembly 102 (such as the core element 106, or other electrical wire, etc.) to satisfy current leakage requirements. For instance, a maximum dimension of the core element 106 may be (preferably) about 0.029 inches in order for the outer diameter of the flexible medical guidewire assembly 102 to remain under (preferably) about 0.035 inches. In accordance with an embodiment, the jacket element 108 includes a relatively thicker electrical insulation (for ease of manufacturing). The thickness may be sized to change the effective outer diameter of the flexible medical guidewire assembly 102. For instance, for the case where the core element 106 has a diameter (preferably) of about 0.018 inches, and has a stainless steel material. The jacket element 108 includes a relatively thicker layer of PTFE to bring the outer device diameter of the flexible medical guidewire assembly 102 to (preferably) about 0.035 inches. In accordance with an embodiment, the sensor device (medical sensor, electrode, etc.) is positioned to the flexible medical guidewire assembly 102. For instance, the sensor device may be configured for low-voltage electrical communication (such as for an ECG recording), and a relatively thicker insulation is not required, and may be protected from a primary energy source and interference (preferably, end to end). In accordance with an embodiment, the flexible medical guidewire assembly 102 may be biocompatible, maneuverable, and robust.

FIG. 2A and FIG. 2B depict front views of embodiments of the flexible medical guidewire assembly 102 of FIG. 1A, FIG. 1B, FIG. 1C and/or FIG. 1D.

Referring to the embodiment as depicted in FIG. 2A, a signal measurement system 904 (also called a signal analysis system) is configured to be electrically connectable (selectively electrically connectable, coupled) to a sensor-interface system 906. The definition of “electrically connected” includes electro-magnetically connected, magnetically connected, acoustically connected, photonically connected, etc. The signal measurement system 904 may include, for instance, an electromagnetic system, an electroanatomic mapping system (3D (three dimensional) or 2D (two dimensional)), or an electroanatomic nonfluoroscopic mapping system, etc. The sensor assembly 104 includes (for instance) the magnetic sensor device 402 (in accordance with and as depicted in FIG. 2A). The sensor-interface system 906 is configured to interface with the sensor assembly 104. The sensor-interface system 906 is configured to exchange (receive and/or transmit) signals (information) with the sensor assembly 104 (once the sensor-interface system 906 is interfaced with the sensor assembly 104, and once the sensor assembly 104 is activated). The exchange of signals may include having the sensor-interface system 906 transmit a signal to the sensor assembly 104 and/or receive a signal from the sensor assembly 104. For the embodiment as depicted in FIG. 2A, the sensor assembly 104 includes a magnetic device, and the sensor-interface system 906 is configured to magnetically interact with the sensor assembly 104.

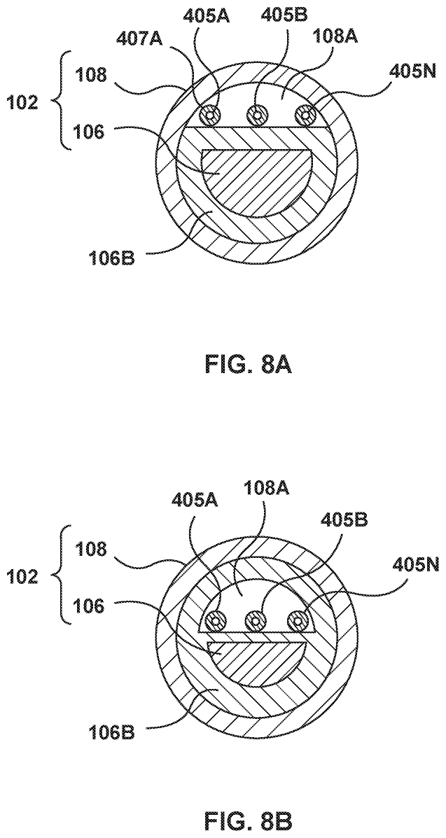

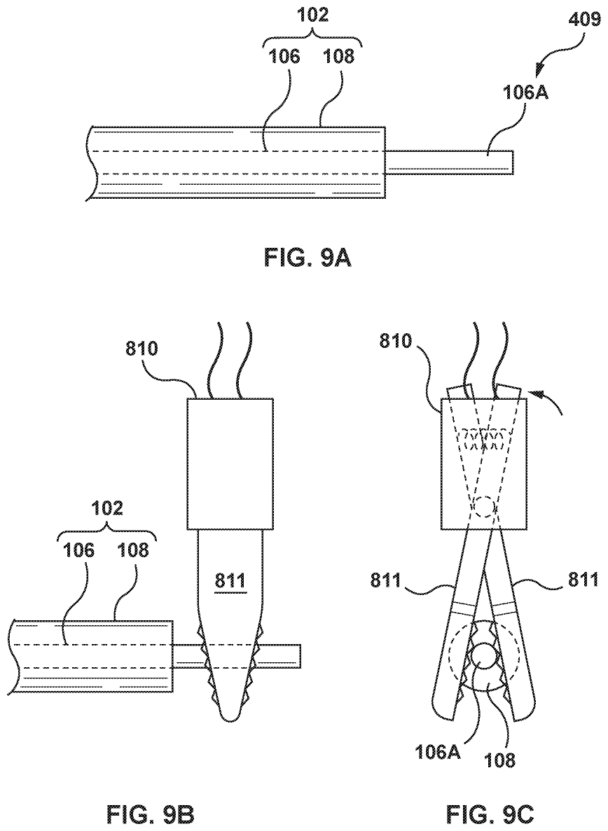

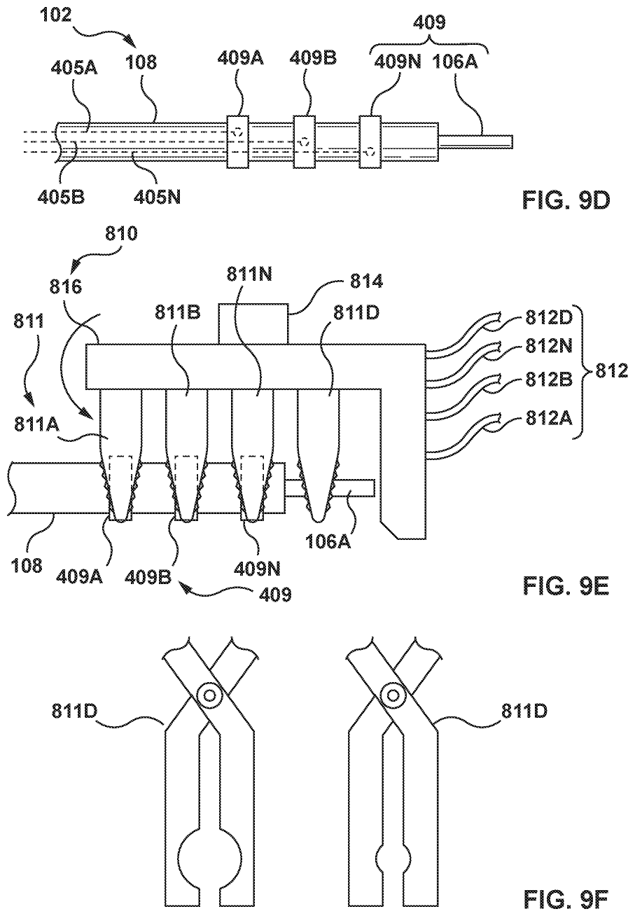

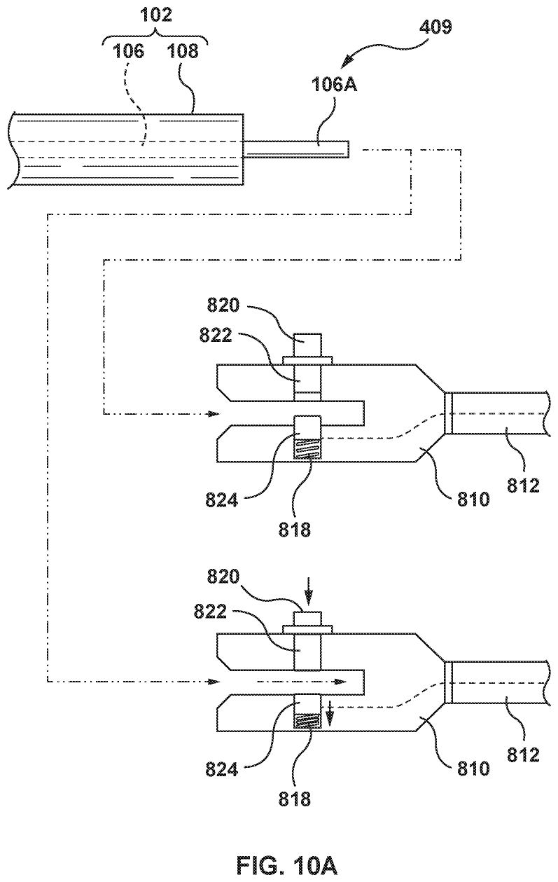

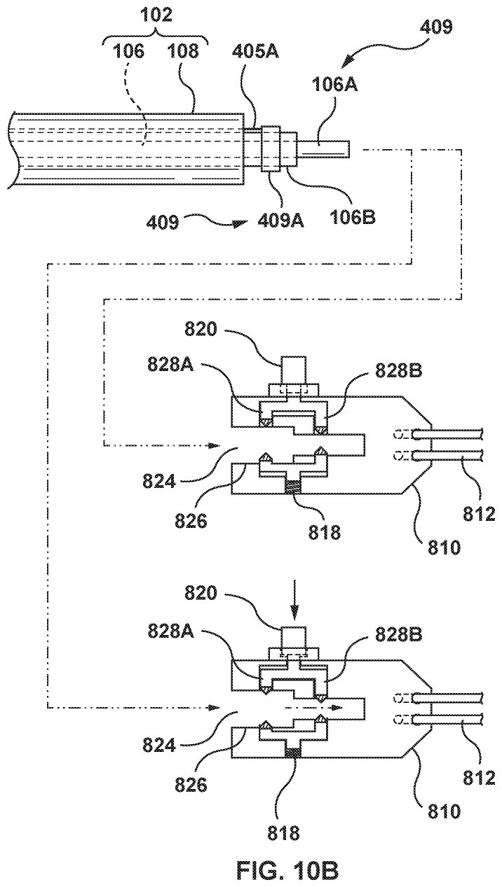

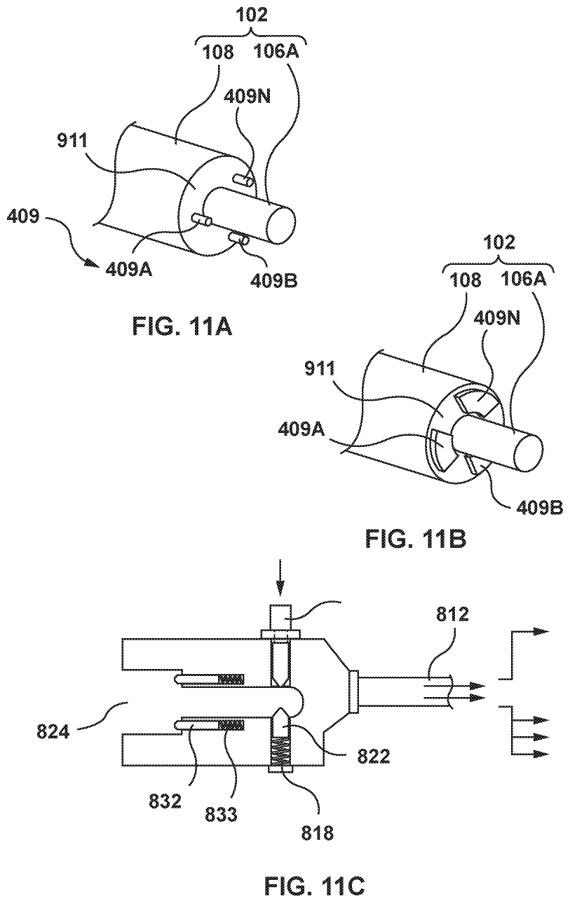

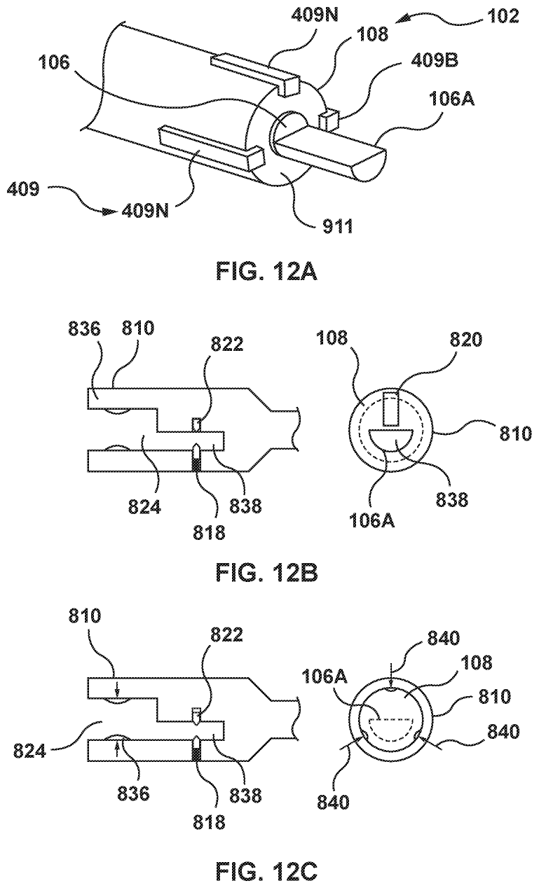

Referring to the embodiment as depicted in FIG. 2B, the flexible medical guidewire assembly 102 includes the electrical wire 405 (not depicted in FIG. 2B but is depicted in the embodiment of FIG. 1C). The sensor assembly 104 includes (preferably) the electrical sensor device 404. The flexible medical guidewire assembly 102 includes an electrical connector 810. Embodiments of the electrical connector 810 are depicted in FIG. 9B, FIG. 9C, FIG. 9D, FIG. 9E, FIG. 9F, FIG. 10A, FIG. 10B, FIG. 11A, FIG. 11B, FIG. 11C, FIG. 12A, FIG. 12B and FIG. 12C. The electrical connector 810 is configured to be electrically connected to (selectively connected to and disconnected from) the electrical wire 405 of the flexible medical guidewire assembly 102. The electrical connector 810 is configured to be electrically connected to (selectively connected to and disconnected from) the sensor-interface system 906. The sensor-interface system 906 is configured to condition the signal received from the sensor assembly 104, and to (then) provide the conditioned signal to the signal measurement system 904 (also called a signal analysis system). In accordance with one option, the sensor-interface system 906 is configured to be electrically connected to (selectively connected to and disconnected from) the signal measurement system 904. In accordance with another option, the sensor-interface system 906 is electrically connected to the signal measurement system 904. The electrical connector 810 is configured to be in electrical communication with the sensor assembly 104 (such as, via the electrical wire 405 as depicted in FIG. 1C) once connected thereto (operatively connected thereto). The electrical connector 810 is also configured to be electrically connectable with the sensor-interface system 906. Preferably, a grounding element 908 (a grounding pad) is configured to be in physical contact with the living body 902 (preferably removably adhered to or in intimate detachable contact with) the skin of the living body 902). The grounding element 908 is spaced apart from the sensor assembly 104. The grounding element 908 is (preferably) positioned proximate to the sensor assembly 104 (at a zone of interest for obtaining signals via the sensor assembly 104). The sensor-interface system 906 is configured to electrically communicate with the sensor assembly 104 once (A) the sensor-interface system 906 is electrically connected to the electrical wire 405 (via the electrical connector 810), and (B) the grounding element 908 is placed in imitate physical contact with the living body 902.

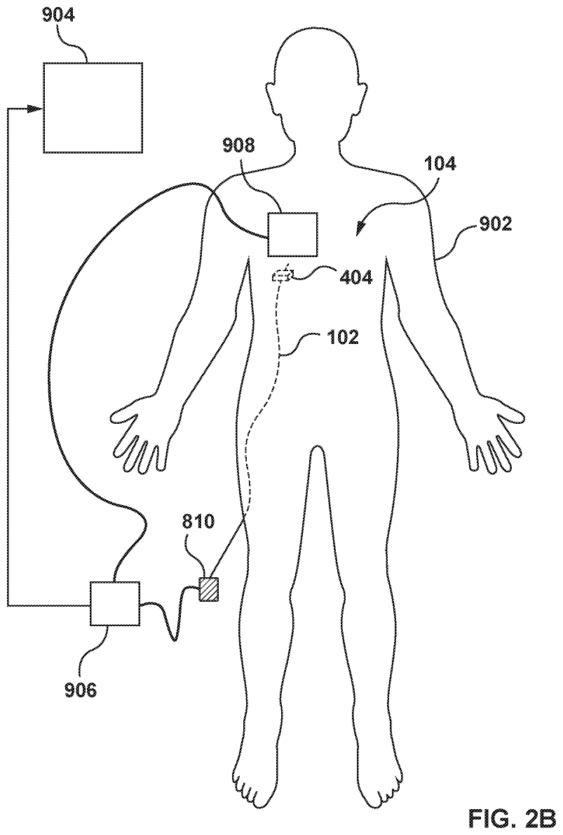

FIG. 3 depicts a front perspective view of an embodiment of the flexible medical guidewire assembly 102 of FIG. 1D (in which some of the technical features as depicted in FIG. 3 may be applicable to the embodiments as depicted in FIG. 1A, FIG. 1B and FIG. 1C where applicable).

Referring to the embodiment as depicted in FIG. 3, the flexible medical guidewire assembly 102 is (in accordance with a preferred embodiment) configured to provide (include) the heating device 112. The heating device 112 is configured to emit RF (radio frequency) energy from (a distal end of) the flexible medical guidewire assembly 102 and into the adjacently positioned tissue of the living body 902 (as depicted, for instance, in the embodiment of FIG. 2B). The core element 106 provides a relatively stiff and electrically conductive material (positioned along the central radial axis extending along the length of the flexible medical guidewire assembly 102). The jacket element 108 includes an electrically insulated material (preferably a polymer layer) covering the core element 106. The jacket element 108 (preferably) further includes an outer polymer layer covering the jacket element 108 (if desired). For instance, the jacket element 108 may include a plastic material having electrical insulation properties suitable for wiring, cabling and/or electrical shielding duties with a sufficient performance properties (dielectric strength, thermal performance, insulation and corrosion, water and heat resistance) for safe performance to comply with industrial and regulatory safety standards. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014]. The core element 106 terminates into (and electrically connects with) the heating device 112 (also called a blunt dome-shaped electrode) positioned at a distal end of the flexible medical guidewire assembly 102. The heating device 112 is configured to puncture the tissue of the living body 902 (once activated accordingly). The core element 106 further includes a core terminal portion 106A extending from the proximal end of the core element 106. The core terminal portion 106A is exposed (not covered, at least in part, with electrical insulation material). The core terminal portion 106A is configured to be electrically connected to an auxiliary equipment (known and not depicted, such as an RF generator, etc.). The flexible medical guidewire assembly 102 (as depicted in FIG. 3) is configured for performing (forming) an RF (radio frequency) puncture (useful for forming a transseptal puncture, etc.) into the tissue of the patient. A technical effect or technical advantage of the heating device 112 is that the heating device 112 may reduce the number of medical device exchanges (insertion and removal of the flexible medical guidewire assembly 102 with the confined space defined by the living body 902), as may be required, for instance, for TSP (transseptal puncture) and cardiac catheterization (requiring the heating device 112). It may be in the interest of both the patient (the living body 902) and the physician to minimize medical device exchanges in the body of the patient to mitigate risk of unwanted medical occurrences or situations, such as air embolism, etc. For the preferred embodiment as depicted in FIG. 3, the core element 106 is electrically conductive, and the heating device 112 is electrically connected to the core element 106.

Referring to the embodiment as depicted in FIG. 3, the jacket element 108 is configured to electrically insulate (provide electrical insulation to) the core element 106. In order to provide electrical safety to the patient and the user (such as the doctor or the medical technician handling the flexible medical guidewire assembly 102), and additionally to provide effective current delivery to the heating device 112 (such as a distal electrode, configured for radio frequency (RF) tissue removal and/or tissue cauterization, etc.), the core element 106 (or an equivalent alternative high voltage wire line) may require a significant (sufficient) amount of electrical insulation. PTFE is a preferred material for high voltage RF insulation owing to its relatively higher electrical performance, biocompatibility and flexibility. PTFE is also available as a heat shrink material (format) to ensure conformal adherence to the core element 106 (the electrically energized mandrel) with efficient use of available space in the flexible medical guidewire assembly 102 (i.e. mitigate space consumed by voids between the electrical wires and/or hollow elongated extrusion voids or lumens extending interiorly along a longitudinal length of the flexible medical guidewire assembly 102). The flexible medical guidewire assembly 102 may have an outer diameter of about 0.035 inches, and may require the effective insulation of about 0.003 inches of wall thickness of PTFE material to satisfy the current (amperage) leakage requirements of electrosurgical medical standards. Preferably, the maximum internal diameter of the core element 106 is about 0.029 inches (in accordance with what may permitted within manufacturing tolerances).

Referring to the embodiment as depicted in FIG. 3, the sensor assembly 104 includes at least one electrical sensor.

Referring to the embodiment as depicted in FIG. 3, the sensor assembly 104 (such as an electrical sensor, etc.) includes a first electrical sensor device 404A (also called a proximal electrode) and an Nth electrical sensor device 404N (in which N is any integer from 1, 2, 3, etc.). The first electrical sensor device 404A and the Nth electrical sensor device 404N are spaced apart from each other, and are fixedly positioned along a longitudinal length of the flexible medical guidewire assembly 102 (each of the electrical sensor devices are spaced apart from each other). In accordance with a preferred option, the first electrical sensor device 404A and the Nth electrical sensor device 404N are (preferably) placed on the outer surface of the jacket element 108. A first electrical wire 405A is electrically connected to the first electrical sensor device 404A (and so on). The first electrical wire 405A is embedded within the flexible medical guidewire assembly 102. The first electrical wire 405A extends along a length of the flexible medical guidewire assembly 102 from the first electrical sensor device 404A to the proximal end of the flexible medical guidewire assembly 102 (terminal end), and so on for the remaining electrical wires. An Nth electrical wire 405N (Nth electrical wire) is electrically connected to the Nth electrical sensor device 404N. The Nth electrical wire 405N (Nth electrical wire) extends along the length of the flexible medical guidewire assembly 102 from the Nth electrical sensor device 404N to the proximal end of the flexible medical guidewire assembly 102 (terminal end). The Nth electrical wire 405N is embedded within the flexible medical guidewire assembly 102. The jacket element 108 (electrical insulation layer) covers and electrically insulates the first electrical wire 405A and the Nth electrical wire 405N. The first electrical wire 405A and the Nth electrical wire 405N are aligned parallel with the core element 106. The first electrical wire 405A and the Nth electrical wire 405N terminate at the core terminal portion 106A. In accordance with a preferred embodiment (as depicted in FIG. 3), the heating device 112 and the plurality of electrical sensor devices (404A, 404N) are electrically insulated from each other (in order to avoid any electrical and/or magnetic interferences between these devices). Reference is made to the following publication for consideration in the selection of a suitable material for electrical insulation: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiment as depicted in FIG. 3, the core terminal portion 106A (proximal electrical connection) is located at a terminal end of the core element 106. The core terminal portion 106A is exposed for electrical connection to a measuring system (via electrical wiring), as depicted in the embodiment of FIG. 2B. Preferably, the core terminal portion 106A extends from (such as, axially extends from) an end section of the core element 106.

Referring to the embodiment as depicted in FIG. 3, the electrical sensor devices (404A, 404N) may include conductive rings configured to fit over the outer surface of the jacket element 108.

The electrical sensor devices (404A, 404N) may be swaged or glued to the outer surface of the flexible medical guidewire assembly 102.

The electrical sensor devices (404A, 404N) may include gold and/or steel.

Referring to the embodiment as depicted in FIG. 3, the sensor assembly 104 (electrical sensor device) may include at least one exposed portion of the electrical wire 405, such as portions of the electrical wires (405A, 405N) that are exposed to (or positioned on) the outer surface of the flexible medical guidewire assembly 102. The exposed portion of the electrical wire 405 is exposed without having the jacket element 108 positioned overtop of the exposed portion of the electrical wire 405. In this manner, the exposure of the electrical wire 405 to the tissue (for instance, the bloodstream of the patient) may provide electrical communication and a signal sensing function for utilization of other medical equipment (such as, an electrocardiography machine, etc.). It will be appreciated that for the case where the electrical wire 405 persists interiorly of, and along the length of, the flexible medical guidewire assembly 102, a window is formed through the jacket element 108 that may expose the electrical wire 405 which acts as the sensor assembly 104 (an electrode, etc.). In this manner, the sensor assembly 104 may include the exposed portion of the electrical wire 405 (that is, the electrical wire 405 is exposed to the exterior of the jacket element 108).

Referring to the embodiment as depicted in FIG. 3, the sensor assembly 104 includes (in accordance with an embodiment) an exposed portion of an electrical wire 405 exposed to an outer surface of the flexible medical guidewire assembly 102. The electrical wire 405 extends along a length of the flexible medical guidewire assembly 102. The electrical wire 405 extends toward a terminal end of the flexible medical guidewire assembly 102. The electrical wire 405 terminates at (is electrically connected to) a terminal contact positioned or located at an end portion of the flexible medical guidewire assembly 102.

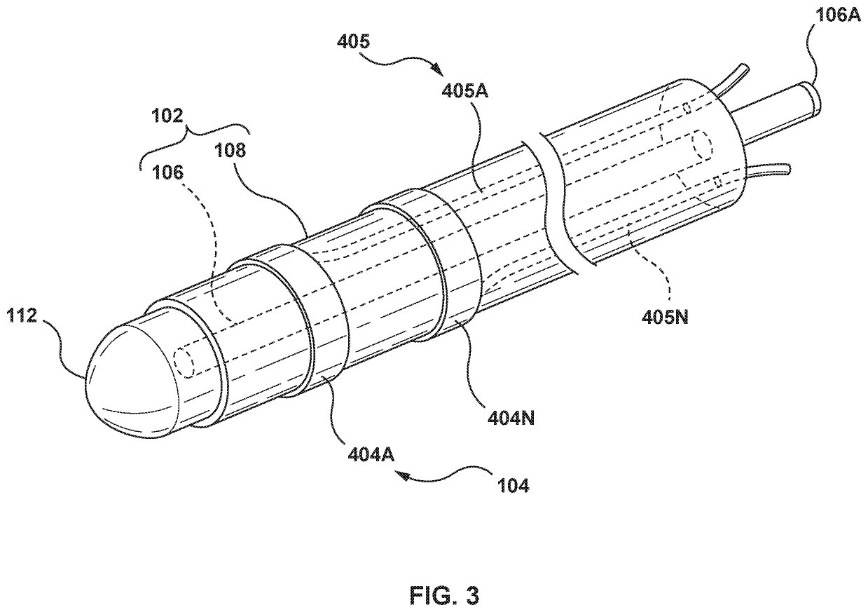

FIG. 4 depicts a front perspective view of an embodiment of the flexible medical guidewire assembly 102 of FIG. 1D (in which some of the technical features as depicted in FIG. 4 may be applicable to the embodiments as depicted in FIG. 1A, FIG. 1B and FIG. 1C where applicable).

Referring to the embodiment as depicted in FIG. 4, the first electrical sensor device 404A and the Nth electrical sensor device 404N are counter sunk (positioned) below the outer diameter (outer surface) of the jacket element 108. The technical effect of this arrangement is to permit relatively easier sliding movement of the flexible medical guidewire assembly 102 along the confined space defined by the living body 902 (as depicted in the embodiment of FIG. 2A or FIG. 2B). In this embodiment (as depicted in FIG. 4), the first electrical sensor device 404A and the Nth electrical sensor device 404N are configured to communicate signals, via the electrical wires (405A, 405N), to the outside environment (for electrical recordings, etc., by external medical equipment) through spatially-separated windows (voids) formed in (on) the outermost layer of the jacket element 108.

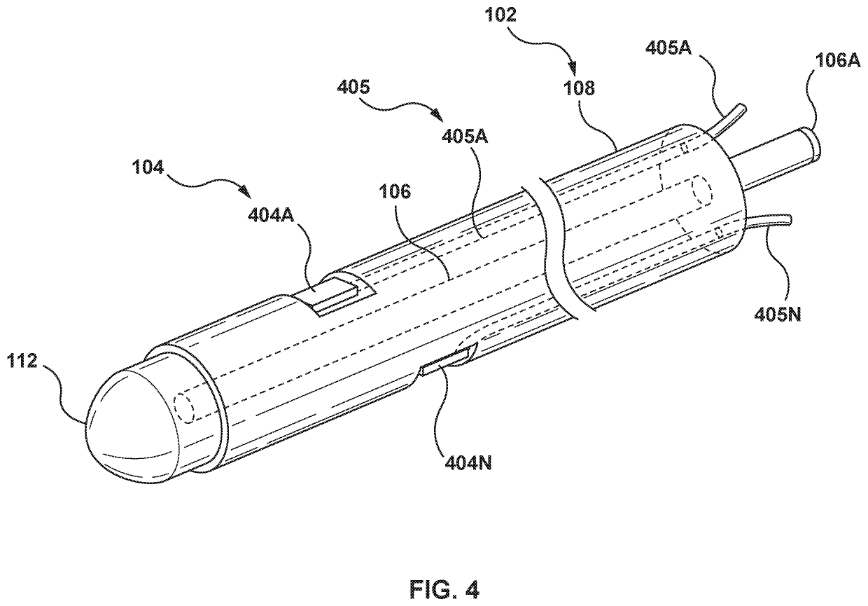

FIG. 5 depicts a front perspective view of an embodiment of the flexible medical guidewire assembly 102 of FIG. 1D (in which some of the technical features as depicted in FIG. 5 may be applicable to the embodiments as depicted in FIG. 1A, FIG. 1B and FIG. 1C where applicable).

Referring to the embodiment as depicted in FIG. 5, the flexible medical guidewire assembly 102 further includes (and is not limited to) a braided element 406.

The braided element 406 is positioned interiorly within the body of the flexible medical guidewire assembly 102. The braided element 406 may include a metal alloy. The braided element 406 is resiliently flexible (resiliently deformable). The braided element 406 includes a braided wire having strands of wire braided together. The braided element 406 is positioned below the outer surface of the flexible medical guidewire assembly 102. The braided element 406 is spaced apart from (and surrounds) the core element 106. The braided element 406 is configured to improve the stiffness and/or torquability of the flexible medical guidewire assembly 102. For instance, for the case where it is necessary to minimize the diameter of the flexible medical guidewire assembly 102 (thus, the stiffness of the core element 106), the braided element 406 may recover some of the stiffness of the flexible medical guidewire assembly 102.

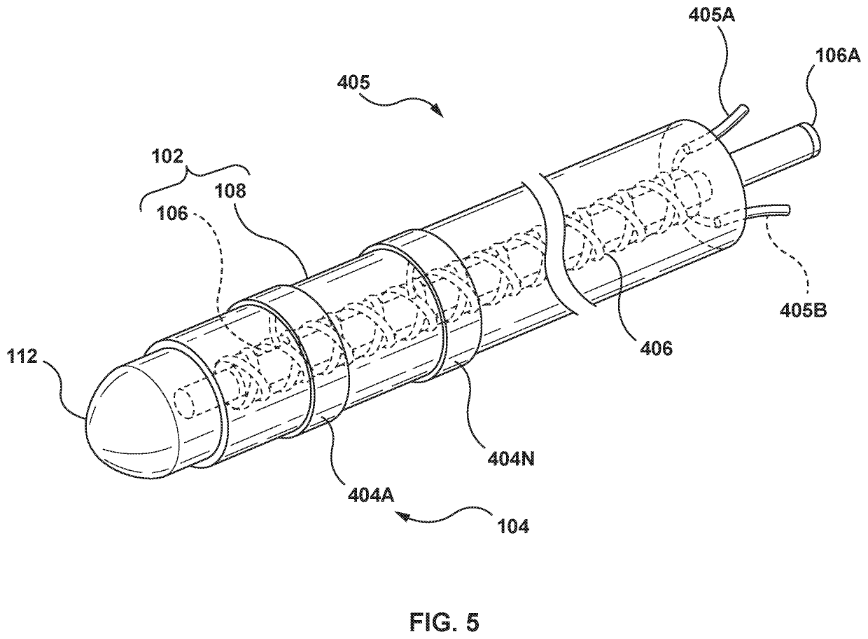

FIG. 6A and FIG. 6B depict an axial cross-sectional view (FIG. 6A) and a radial cross-sectional view (FIG. 6B) of embodiments of the flexible medical guidewire assembly 102 of FIG. 1D (in which some of the technical features as depicted in FIG. 6A and/or FIG. 6B may be applicable to the embodiments as depicted in FIG. 1A, FIG. 1B and FIG. 1C where applicable).

Referring to the embodiment as depicted in FIG. 6A and FIG. 6B, the stiffness of the flexible medical guidewire assembly 102 is (preferably) provided by the electrical wire, such as a combination of electrical wires (the first electrical wire 405A, the second electrical wire 405B and the Nth electrical wire 405N), and is not provided by the core element 106 (or the core element is 106 is not provided as an alternative). That is, the first electrical wire 405A, the second electrical wire 405B and the Nth electrical wire 405N are made relatively larger in size (to add more stiffness to the flexible medical guidewire assembly 102); the core element 106 may be included, or is not included, with the flexible medical guidewire assembly 102. For the case where the core element 106 is included (deployed), the core element 106 may be reduced in diameter to improve manufacturability and positioning of the first electrical sensor device 404A (proximal electrodes) and the Nth electrical sensor device 404N (proximal electrodes), etc. A core insulation layer 106B is positioned over the core element 106. A jacket portal 108A (void) is formed within the central zone of the jacket element 108, so that the core element 106, the first electrical wire 405A, the second electrical wire 405B and the Nth electrical wire 405N are all positioned in the jacket portal 108A. A technical effect of this embodiment is that once the flexible medical guidewire assembly 102 is made to curve, the electrical wires may flex and bend without electrically breaking or shorting, etc.

In accordance with the embodiments as depicted in FIG. 6A and FIG. 6B, the diameter of the core element 106 (also called the primary mandrel) is (preferably) reduced. For instance, the core element 106 includes an elongated wire (a relatively thin wire, for lower voltage capability) extending along a longitudinal length of the flexible medical guidewire assembly 102. The core element 106 includes a relatively lower surface area and/or diameter, and this arrangement provides additional room for other components (for placement within and along an elongated length of the jacket element 108 (also called an insulation layer). The outer dimension (diameter) of the first electrical wire 405A may be increased to provide increased mechanical stiffness for the flexible medical guidewire assembly 102. To balance the stiffness profile of the flexible medical guidewire assembly 102 (such as, to provide a relatively floppier tip portion, a relatively stiffer proximal body portion, etc.), the profiles (outer diameters) of the core element 106 and/or the electrical wire 405 may vary along a longitudinal length of the flexible medical guidewire assembly 102. For instance, a relatively longer instance of the of the core element 106 may include a reduction in outer diameter and a cross sectional area, along their length, to provide a desired flexibility (regional flexibility) for selected portion of the flexible medical guidewire assembly 102. For instance, the outer diameter of the core element 106 may increase towards the distal tip (of the flexible medical guidewire assembly 102) to improve fixation and stiffness at a distal most positioned medical device (such as a medical sensor device).

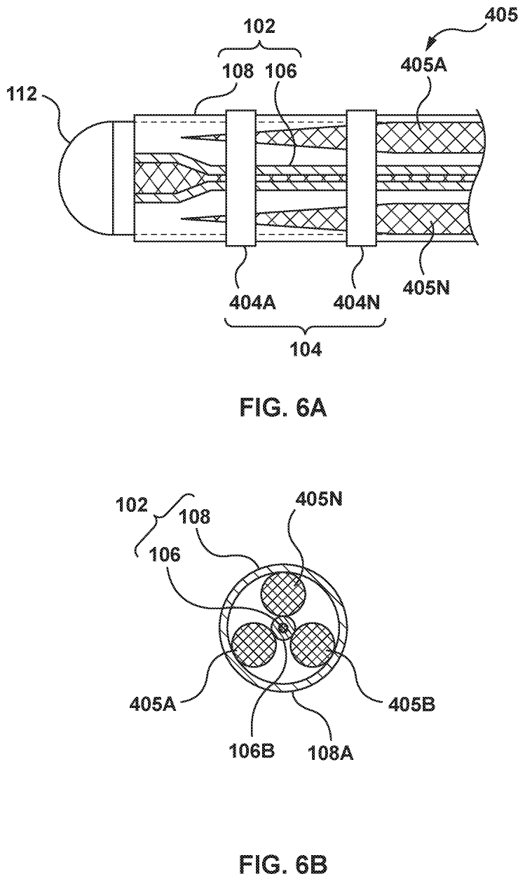

FIG. 7A, FIG. 7B, FIG. 7C, FIG. 7D and FIG. 7E depict radial cross-sectional views of embodiments of the flexible medical guidewire assembly 102 of FIG. 1D (in which some of the technical features as depicted in FIG. 7A, FIG. 7B, FIG. 7C, FIG. 7D and/or FIG. 7E may be applicable to the embodiments as depicted in FIG. 1A, FIG. 1B and FIG. 1C where applicable).

Referring to the embodiment as depicted in FIG. 7A, the first wire insulation layer 407A is placed over the first electrical wire 405A, and so on for each electrical wire. The second wire insulation layer 407B is placed over the second electrical wire 405B. The Nth wire insulation layer 407N is placed over the Nth wire insulation layer 407N. The core element 106 has an outer diameter that is larger than the outer diameters of the first electrical wire 405A, the second electrical wire 405B and the Nth electrical wire 405N. The core element 106, the first electrical wire 405A, the second electrical wire 405B and the Nth electrical wire 405N are located within the jacket portal 108A, and are arranged coaxially along a length (longitudinal axis) of the flexible medical guidewire assembly 102. In accordance with an embodiment, the core element 106, the first electrical wire 405A, the second electrical wire 405B and the Nth electrical wire 405N are (preferably) electrically insulated from each other (and from the patient, etc.). In accordance with another embodiment, the first electrical wire 405A, the second electrical wire 405B and the Nth electrical wire 405N are (preferably) electrically insulated from each other and from the patient (for the case where the core element 106 is not electrically conductive). The flexible medical guidewire assembly 102 has (preferably) an outer diameter from about 0.032 inches to about 0.035 inches. The core element 106 has or includes (preferably) an outer diameter of about 0.018 inches (preferably, of SAE 304 spring tempered stainless steel). The electrical insulation layer (such as the core insulation layer 106B as depicted in FIG. 6B, also called the primary insulation) is positioned over the core element 106, and has (preferably) a thickness of about 0.003 inches. The electrical wires (405A, 405B, 405N, etc.) may have any suitable cross section or profile, such as a round cross section or a rectangular cross section. The electrical wires (405A, 405B, 405N, etc.) may have a thickness dimension (diametric contribution) of about 0.002 inches. The wire insulation layer placed over each of the electrical wires (405A, 405B, 405N, etc.) may have a thickness of about 0.003 inches (or greater).

There is sufficient space in or on the flexible medical guidewire assembly 102 to permit placement of the sensor devices (also called ring electrodes as depicted in FIG. 6A, for instance). In accordance with an embodiment, the sensor devices (proximal electrodes) may be placed (positioned) in a relatively floppy section of the flexible medical guidewire assembly 102 where the outer diameter of the core element 106 has been reduced to improve floppiness (thereof). A floppy region (of the flexible medical guidewire assembly 102) may include a stainless steel material (an elongated mandrel) that ranges from about 0.006 inches to about 0.010 inches (in diameter). For this case, the diametric constraints are reduced to provide additional space to terminate the connectors to the sensor devices (proximal electrodes), etc.

Referring to the embodiment as depicted in FIG. 7B, the core insulation layer 106B is positioned over the core element 106. The jacket portal 108A is located between the outer surface of the core insulation layer 106B and the inner surface of the jacket element 108. The first electrical wire 405A, the second electrical wire 405B and the Nth electrical wire 405N are positioned in the jacket portal 108A (between the jacket element 108 and the core insulation layer 106B). The jacket portal 108A forms a multisided geometric shape (having, for instance, a multi-angled cross section with the electrical wires positioned at the vertices of the multisided geometric shape (such as a triangle)).

Referring to the embodiment as depicted in FIG. 7C, the core insulation layer 106B defines recesses configured to receive a respective electrical wire (405A, 405B, 405N) therein. The outer shape of the core insulation layer 106B has a round or circular cross-sectional shape. The inner surface shape of the jacket element 108 has a round or circular cross-sectional shape that conforms the outer shape of the core insulation layer 106B. The core insulation layer 106B surrounds the core element 106.

Referring to the embodiment as depicted in FIG. 7D, this embodiment provides a similar embodiment as depicted in FIG. 7B with the electrical wires (405A, 405B, 405N) each having a respective electrical insulation layer thereon (as depicted in the embodiment of FIG. 7A).

Referring to the embodiment as depicted in FIG. 7E, the jacket element 108 defines (provides) jacket channels (a first jacket channel 109A, a second jacket channel 109B and an Nth jacket channel 109N). Each of the electrical wires (405A, 405B, 405N) are respectively received in a respective jacket channel (109A, 109B, 109N). It will be appreciated that the core insulation layer 106B is optional in this embodiment.

In accordance with the embodiments as depicted in FIG. 7A to FIG. 7E, an increase in a relative movement between (and/or slack for) the electrical wires (405A, 405B, 405N) may provide additional flexibility for the flexible medical guidewire assembly 102. The core element 106 (also called a mandrel, which may be electrically conductive) is configured to provide a majority of the mechanical stiffness (relative to the jacket element 108 and the electrical wires (405A, 405B, 405N)). The jacket element 108 (also called a coating) is configured to provide sufficient electrical insulation that may withstand a relatively higher voltage and/or current for the core element 106. The electrical wires (405A, 405B, 405N) are electrically insulated (electrically isolated) from each other and the core element 106. The electrical wires (405A, 405B, 405N) may be configured to handle a relatively lower voltage or a relatively higher voltage, etc. The jacket element 108 may be flexible and may be lubricous (may be smooth and slippery with oil or a similar substance).

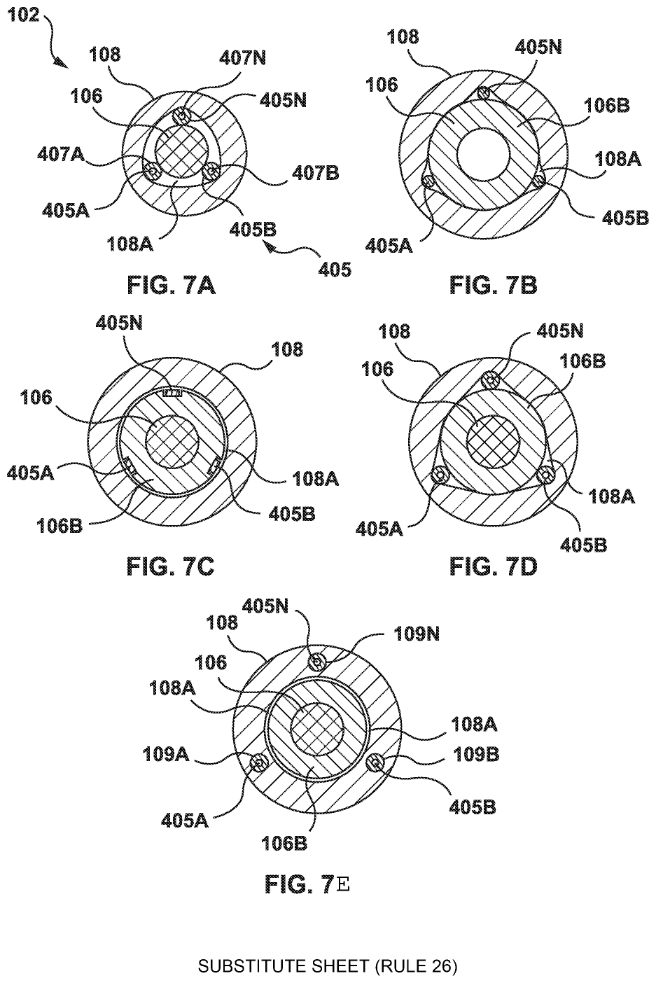

FIG. 8A and FIG. 8B depict radial cross-sectional views of embodiments of the flexible medical guidewire assembly 102 of FIG. 1D (in which some of the technical features as depicted in FIG. 8A and/or FIG. 8B may be applicable to the embodiments as depicted in FIG. 1A, FIG. 1B and FIG. 1C where applicable).

Referring to the embodiment as depicted in FIG. 8A, the jacket element 108 defines (forms) the jacket portal 108A (having a circular cross-sectional profile). The core insulation layer 106B is placed over the core element 106, each having a semicircular cross-sectional profile. The core element 106 forms an offset (non-circular) shape. The core element 106 and the core insulation layer 106B are received in at least a part of the jacket portal 108A, leaving a portion of the jacket portal 108A open and available for receiving the electrical wires (405A, 405B, 405N). Each of the electrical wires (405A, 405B, 405N) have an electrical insulation layer (such as the first wire insulation layer 407A, etc.). The core element 106 forms a shape configured to provide additional space to improve manufacturability and scalability of the electrical wires (405A, 405B, 405N). Referring to the embodiment as depicted in FIG. 8B, the core insulation layer 106B forms a void configured to receive the electrical wires (405A, 405B, 405N). The core insulation layer 106B forms two voids (cavities); one cavity for receiving the core element 106, and the other cavity for receiving the electrical wires (405A, 405B, 405N).

FIG. 9A depicts a side view of an embodiment of the flexible medical guidewire assembly 102 of FIG. 1D (in which some of the technical features as depicted in FIG. 9A may be applicable to the embodiments as depicted in FIG. 1A, FIG. 1B and FIG. 1C where applicable). FIG. 9B, FIG. 9C, FIG. 9D, FIG. 9E and FIG. 9F depict side views of embodiments of an electrical connector 810 configured to be connectable to the flexible medical guidewire assembly 102 of FIG. 9A.

Referring to the embodiment as depicted in FIG. 9A, the core terminal portion 106A extends (extends axially) from an end portion (proximal portion or user accessible portion) of the core element 106. The core terminal portion 106A is exposed (for electrical connection, for the case where the core terminal portion 106A is electrically conductive). The flexible medical guidewire assembly 102 includes at least one terminal portion 409. The terminal portion 409 may include the core element 106 (for the case where it is required for the core element 106 to be electrically conductive).

Referring to the embodiments as depicted in FIG. 9B and FIG. 9C, an electrical connector 810 is configured to be selectively electrically connected to (clipped to) the core terminal portion 106A. The electrical connector 810 may include a clip connector, an alligator clip, etc., and any equivalent thereof. The electrical-connector assembly 810 includes a connector terminal 811 (an electrical connector contact, a jaw portion, etc.) such as a pair of opposed jaws (spring biased to be normally closed, etc.). In accordance with an embodiment, the electrical-connector assembly 810 is configured to connect to the electrical terminals (the terminal portion 409, electrical connections) that are positioned at a proximal end of the flexible medical guidewire assembly 102. The electrical terminals are electrically connected to the electrical sensor device 404A and/or a medical device (such as the heating device 112) as depicted, for instance, in FIG. 3, etc., which are mounted to a portion (such as a distal portion) of the flexible medical guidewire assembly 102. The electrical-connector assembly 810 is also configured to connect the electrical terminals of an external device (such as a signal generator or a signal-recording system, etc., which are known and not depicted) to the electrical terminals (at least one or more instances of the terminal portion 409) of the flexible medical guidewire assembly 102. This is done, preferably, in such a way that the nominal diameter of the flexible medical guidewire assembly 102 may be maintained (such as, from about 0.032 inches to about 0.035 inches, etc.). Preferably, the electrical-connector assembly 810 provides conductive pins configured to allow (facilitate) the electrical connections with the electrical terminals (terminal portion 409) of the flexible medical guidewire assembly 102 with a single user motion (for convenience). Alternatively, the electrical-connector assembly 810 is configured to provide conductive pins configured to allow for (facilitate) the electrical connections between the electrical terminals of the flexible medical guidewire assembly 102 and multiple selective independent connections, etc.

Referring to the embodiment as depicted in FIG. 9D, the core terminal portion 106A extends axially from an end portion of the core element 106. The core terminal portion 106A is exposed for electrical connection (for the case where the core terminal portion 106A is electrically conductive, etc.). At least one terminal portion (409A, 409B, 409N) (also called proximal electrical connectors or wire terminals) are electrically coupled to respective electrical sensor devices (404A, 404N) that are depicted in any one of the embodiments of FIG. 3, FIG. 4 and/or FIG. 5. For instance, a first terminal portion 409A (wire terminal, proximal connection) is electrically coupled to the first electrical sensor device 404A (via the first electrical wire 405A as depicted in FIG. 3). The second terminal portion 409B (proximal electrode) is electrically coupled to the second electrical sensor device (not depicted but easy to visualize given the first electrical sensor device 404A) via the second electrical wire 405B. The Nth terminal portion 409N (proximal connection) is electrically coupled to the Nth electrical sensor device 404N (via the Nth electrical wire 405N). The terminal portions (409A, 409B, 409N) are spaced apart (axially spaced apart) from each other along a length of the jacket element 108, and are positioned on the outer surface of the jacket element 108 proximate to the end portion of the flexible medical guidewire assembly 102 (near the location of the core terminal portion 106A). In accordance with a preferred embodiment, the flexible medical guidewire assembly 102 is configured to facilitate catheter exchange by having the proximal end (the user access end portion) with no handle to enable a catheter to pass over a length (an entire length) of the flexible medical guidewire assembly 102; for the case where a handle were to be positioned at the proximal end, then the catheter may not be able to pass over a length of the flexible medical guidewire assembly 102. In accordance with a preferred embodiment, the flexible medical guidewire assembly 102 includes multiple instances of the sensor assembly 104 in which the sensor assemblies 104 are connectable to a measuring device (as depicted in FIG. 2A or FIG. 2B).

Referring to the embodiment as depicted in FIG. 9E and FIG. 9F, the electrical-connector assembly 810 has a connector terminal 811. The connector terminal 811 is configured to be selectively electrically connectable with (and selectively electrically disconnectable from) at least one terminal portion 409 of the flexible medical guidewire assembly 102. The flexible medical guidewire assembly 102 is configured to be inserted into the confined space defined by a living body 902 (as depicted in FIG. 2A or FIG. 2B). The terminal portion 409 (also called a wire terminal) is electrically connected, via an electrical wire 405, to a sensor assembly 104 of (supported by) the flexible medical guidewire assembly 102 (as depicted in FIG. 1C or FIG. 1D, etc.).

Referring to the embodiment as depicted in FIG. 9E and FIG. 9F, the electrical-connector assembly 810 is configured to (preferably) selectively electrically connect to (and selectively electrically disconnect from) the terminal portions (409A, 409B, 409N, 106A) (electrical terminals, exposed electrical terminals) of the flexible medical guidewire assembly 102. The terminal portions may include, for instance, the core terminal portion 106A (for the case where the core terminal portion 106A is electrically conductive,) and the terminal portions (409A, 409B, 409N) (also called electrical portions, a plurality of proximal electrodes, etc., which may be utilized for the sensor devices, etc.). For the case where the core terminal portion 106A is not required (is not deployed as an electrical wire), the electrical-connector assembly 810 is configured to selectively electrically connect to (and selectively electrically disconnect from) the terminal portions (409A, 409B, 409N) or at least one or more terminal portions connected with at least one electrical sensor device 404 (as depicted in FIG. 3). The electrical-connector assembly 810 includes a connector wire 812, such as connector wires (812A, 812B, 812D, 812N). The connector wire 812 is (preferably) long enough to provide a sufficient amount of slack for spatial movement of the flexible medical guidewire assembly 102 (that is, to provide breakouts to ancillary medical equipment, such as a radio frequency generator, a signal recording system, etc.). The connector wire 812 includes (preferably) a plurality of connector wires (812A, 812B, 812D, 812N). Selective disconnection and removal of the electrical-connector assembly 810 from the terminal portions (409A, 409B, 409N, 106A) of the flexible medical guidewire assembly 102 permits the flexible medical guidewire assembly 102 to be utilized with the medical instrument 900 (as depicted in FIG. 1), and/or with other medical devices, such as a catheter (such as for catheter exchange duties), etc. Referring to the embodiment as depicted in FIG. 9E and FIG. 9F, the electrical-connector assembly 810 has the connector terminal 811 (such as, the first connector terminal 811A, the second connector terminal 811B, and the Nth connector terminal 811N). The connector terminal 811 is configured to be electrically connectable with at least one terminal portion 409 of the flexible medical guidewire assembly 102. The flexible medical guidewire assembly 102 is configured to be inserted into the confined space defined by the living body 902 (as previously described and depicted in FIG. 2A or FIG. 2B)). The terminal portion 409 is exposed (electrically exposed) for electrical connection to the connector terminal 811. The terminal portion 409 (electrical terminal) is electrically connected, via an electrical wire 405, to a sensor assembly 104 (as depicted, for instance, in the embodiment of FIG. 3). The terminal portion 409 is positioned at a proximal end of the flexible medical guidewire assembly 102. The terminal portion 409 is exposed for selective electrical connection to the electrical-connector assembly 810. The sensor assembly 104 and the electrical wire 405 are supported by the flexible medical guidewire assembly 102 (this is done in such a way that the sensor assembly 104 and the flexible medical guidewire assembly 102 are movable along the confined space defined by the living body 902 once the flexible medical guidewire assembly 102 is inserted into, and moved along, the confined space defined by the living body 902).

Referring to the embodiment as depicted in FIG. 9E and FIG. 9F, the connector terminal 811 (of the electrical-connector assembly 810) includes (preferably) connector terminals (811A, 811B, 811N, 811D) for respective terminal portions (409A, 409B, 409N, 106A). The core terminal portion 106A may be utilized or deployed as an electrical wire for the case where a heater device or other medical device is deployed in or with the flexible medical guidewire assembly 102. For instance, the connector terminals (811A, 811B, 811N, 811D) preferably include a first pair of jaws (for electrical connection with the first terminal portion 409A), a second pair of jaws (for electrical connection with the second terminal portion 409B), an Nth pair of jaws (for electrical connection with the Nth terminal portion 409N), and a pair of core jaws (for electrical connection with the core terminal portion 106A). The connector wires (812A, 812B, 812D, 812N) (connector electrical wires) are respectively electrically connected to the connector terminals (811A, 811B, 811N, 811D). The first connector wire 812A is electrically connected to the first connector terminal 811A. The second connector wire 812B is electrically connected to the second connector terminal 811B. The Nth connector wire 812N is electrically connected to the Nth connector terminal 811N. The fourth connector wire 812D is electrically connected to the third connector terminal 811D (for connection to the core terminal portion 106A).

Referring to the embodiment as depicted in FIG. 9E and FIG. 9F, the electrical-connector assembly 810 includes side-loading removable electrical connectors. The electrical-connector assembly 810 includes, for instance, and supports multiple spaced-apart alligator clips that are mounted to (or extend from) the electrical-connector assembly 810.

Referring to the embodiment as depicted in FIG. 9F, the electrical-connector assembly 810 includes an asymmetric hairclip style connector having a hard-stop and variable gaps formed in the alligator teeth for mitigating misconnection and ensuring correct electrical connection to the electrical sensor devices (404A, 404N) as depicted in FIG. 3. The connector terminal 811A is keyed for keyed connection to the first terminal portion 409A. The third connector terminal 811D (such as a jaw) is keyed for keyed connection to the core terminal portion 106A. For the case where the core terminal portion 106A is not deployed as an electrical wire, the third connector terminal 811D is not utilized, etc.

Referring to the embodiment as depicted in FIG. 9F, the electrical-connector assembly 810 includes a handle 814 extending from a housing assembly 816. The first connector terminal 811 is supported by the housing assembly 816. The connector wire 812 is supported by the housing assembly 816.

FIG. 10A and FIG. 10B depict side views of embodiments of an electrical connector 810 configured to be connectable to the flexible medical guidewire assembly 102 of FIG. 1D (in which some of the technical features as depicted in FIG. 10A and/or FIG. 10B may be applicable to the embodiments as depicted in FIG. 1A, FIG. 1B and FIG. 1C where applicable).

Referring to the embodiment as depicted in FIG. 10A, the electrical-connector assembly 810 is configured to electrically interface (interact) with an end portion (proximal end portion) of the flexible medical guidewire assembly 102. The electrical-connector assembly 810 is configured to back load (back connect) to the flexible medical guidewire assembly 102. The electrical-connector assembly 810 defines (provides) a connector channel 824 configured to receive (at least in part, axially receive) a length of the end portion of the flexible medical guidewire assembly 102. The electrical-connector assembly 810 includes a push button 820 that is positioned on a surface (such as a top surface) of the electrical-connector assembly 810. The electrical-connector assembly 810 includes a connector conductor 822 (an electrode, etc.). The push button 820 is configured to (A) be activated by the user (such as the doctor, etc.), which selectively moves the connector conductor 822, and (B) selectively connect the connector conductor 822 with the terminal portions of the flexible medical guidewire assembly 102 (such as the core terminal portion 106A) once the end portion of the flexible medical guidewire assembly 102 is received in the connector channel 824 (and once the push button 820 is activated accordingly).

Referring to the embodiment as depicted in FIG. 10A, the electrical-connector assembly 810 is also configured to provide an over-the-wire connector. The upper instance of the electrical-connector assembly 810 is depicted in the loading position (unactivated), in which the push button 820 is not depressed or not activated by the user (such as the doctor). The lower instance of the electrical-connector assembly 810 is depicted in a locked and engaged position, in which the push button 820 is engaged or activated. For the upper instance of the electrical-connector assembly 810, the push button 820 is ready to be depressed (by the user or doctor). Once the push button 820 is depressed (as shown in the lower instance of the electrical-connector assembly 810), with assistance by the spring member 818, the connector conductor 822 becomes clamped to (electrically engaged with) the terminal portion 409 (such as the core terminal portion 106A). It will be appreciated that the same mechanism may be utilized for the case where there are multiple instances of the terminal portion 409. Each unique electrical connection (i.e., for each instance of the terminal portion 409) may require independent travel to ensure full connection with each connector face to a respective terminal portion of the flexible medical guidewire assembly 102. Alternatively, biased connection to the terminal portion 409 (such as, the core terminal portion 106A) may ensure connection is possible only after other terminal portions (of the flexible medical guidewire assembly 102) have made electrical contact, etc., with the electrical components of the electrical-connector assembly 810.

Referring to the embodiment as depicted in FIG. 10B, the upper instance of the electrical-connector assembly 810 is depicted in the loading position (that is, the electrical-connector assembly 810 is ready to receive the end portion of the flexible medical guidewire assembly 102). The lower instance of the electrical-connector assembly 810 is depicted in a locked and engaged position (the electrical components of the electrical-connector assembly 810 have made electrical contact with the terminals of the flexible medical guidewire assembly 102). The connector channel 824 is formed as a complementary profile 826. The complementary profile 826 has a shape that is complementary to the outer profile of the end portion of the flexible medical guidewire assembly 102. The push button 820 is configured to move (in tandem) the upper electrical terminals including a first pole 828A (for utilization with the first terminal portion 409A), and a second pole 828B (for utilization with the core terminal portion 106A). The first pole 828A and the second pole 828B are spaced apart from each other.

In accordance with the embodiments as depicted in FIG. 10A and FIG. 10B, each unique electrical connection may be provided with independent travel to ensure (full) electrical connection with each connector face (of an electrical wire). Alternatively, a bias connection may be provided the core element 106 to ensure electrical connection is only possible after other poles have made contact.

FIG. 11A and FIG. 11B depict perspective views of embodiments of the flexible medical guidewire assembly 102 of FIG. 1D (in which some of the technical features as depicted in FIG. 11A, FIG. 11B and/or FIG. 11C may be applicable to the embodiments as depicted in FIG. 1A, FIG. 1B and FIG. 1C where applicable). FIG. 11C depicts a side view of an embodiment of an electrical connector 810 configured to be connectable to the flexible medical guidewire assembly 102 of FIG. 11A and/or FIG. 11B.

Referring to the embodiment as depicted in FIG. 11A and FIG. 11B, the end portion (the proximal end or user-accessible portion, etc.) of the flexible medical guidewire assembly 102 includes (provides) a flat radial end face 911. The flat radial end face 911 faces axially along an axial axis that extends outwardly from the end portion of the flexible medical guidewire assembly 102. The first terminal portion 409A includes a first protuberance (an axially extending post) extending axially from (away from) the flat radial end face 911 (that is, extending from the jacket element 108). The second terminal portion 409B includes a second protuberance (an axially extending post) extending axially from (away from) the flat radial end face 911 (that is, extending from the jacket element 108). The Nth terminal portion 409N includes an Nth protuberance (an axially extending post) extending axially from (away from) the flat radial end face 911 (extending from the jacket element 108). The first terminal portion 409A, second terminal portion 409B and Nth terminal portion 409N are respectively electrically connected to an associated electrical sensor device (such as the electrical sensor device 404A as depicted in FIG. 3, etc.), and are spaced apart from each other (preferably, in an equidistant relationship relative to each other). The core terminal portion 106A extends axially from the core element 106 and away from the flexible medical guidewire assembly 102 (along the same direction as the alignment of the terminal portions (409A, 409B, 409N)).

Referring to the embodiment as depicted in FIG. 11B, the end portion (the proximal portion) of the flexible medical guidewire assembly 102 includes a flat radial end face 911 (a back flat face, an end face, etc.) facing along an axial axis extending from the end portion of the flexible medical guidewire assembly 102. The first terminal portion 409A includes a first flattened end face terminal portion extending radially (at least in part) along the flat radial end face 911. The second terminal portion 409B includes a second flattened end face terminal portion extending radially (at least in part) along the flat radial end face 911. The Nth terminal portion 409N includes an Nth flattened end face terminal portion extending (at least in part) radially along the flat radial face 911.

Referring to the embodiment as depicted in FIG. 11C, the electrical-connector assembly 810 is configured to interact with (electrically interface with) the terminal portions (409A, 409B, 409N) of the embodiments as depicted in FIG. 11A and/or FIG. 11B. The terminal portions (409A, 409B, 409N) are positioned at the flat radial end face 911 as depicted in FIG. 11A and/or FIG. 11B. The electrical-connector assembly 810 includes axially-extending wire connections 832 (spring-loaded conductive elements or rods) each having a respective spring member 833. Each respective spring member 833 is configured to bias extension of the axially-extending wire connections 832 outwardly away from the electrical-connector assembly 810. Each respective spring member 833 is positioned within the connector channel 824 defined by the electrical-connector assembly 810.

Referring to the embodiment as depicted in FIG. 11C, the push button 820 is mounted to an exterior of a housing of the electrical-connector assembly 810. The push button 820 is configured to selectively lock to (and selectively unlock from) the end portion (the proximal end) of the flexible medical guidewire assembly 102. It will be appreciated that for some embodiments, the flexible medical guidewire assembly 102 includes the core terminal portion 106A that is electrically conductive with the core element 106 also being electrically conductive, etc. Once the push button 820 is activated (by the user or the doctor, etc.), the push button 820 selectively locks (securely connects) the electrical connector 810 to the end portion of the flexible medical guidewire assembly 102, the radially extending wire connections 832 make electrical contact with the terminal portions (409A, 409B, 409N, 106A), etc. A spring member 818 (also called a compression spring) is positioned in the electrical connector 810, and is configured to bias a connector conductor 822 (across the axial axis of the flexible medical guidewire assembly 102) to the core terminal portion 106A (once the end portion of the flexible medical guidewire assembly 102 is inserted into the connector channel 824 of the electrical connector 810).

FIG. 12A depicts a perspective view of an embodiment of the flexible medical guidewire assembly 102 of FIG. 1D (in which some of the technical features as depicted in FIG. 12A, FIG. 12B and/or FIG. 12C may be applicable to the embodiments as depicted in FIG. 1A, FIG. 1B and FIG. 1C where applicable). FIG. 12B and FIG. 12C depict side views of embodiments of an electrical connector 810 configured to be connectable to the flexible medical guidewire assembly 102 of FIG. 12A.

Referring to the embodiment as depicted in FIG. 12A, the flexible medical guidewire assembly 102 includes (preferably) a plurality of terminal portions (409A, 409B, 409N, 106A) positioned at a terminal end portion (the proximal end portion or user accessible portion) of the flexible medical guidewire assembly 102. In accordance with a preferred embodiment, the plurality of terminal portions (409A, 409B, 409N, 106A) includes the first terminal portion 409A which is electrically connected to the first electrical sensor device 404A as depicted in FIG. 3. The second terminal portion 409B is electrically connected to another electrical sensor device (not depicted). The Nth terminal portion 409N is electrically connected to the Nth electrical sensor device 404N as depicted in FIG. 3. The core terminal portion 106A is electrically connected to the core element 106. At least some of the plurality of terminal portions (409A, 409B, 409N) are mounted to (and extend axially along a portion of) the outer surface of the jacket element 108. At least some of the plurality of terminal portions (409A, 409B, 409N) extend along (at least in part) a radially-extending direction along a flat radial end face 911 (of the flexible medical guidewire assembly 102). The flat radial end face 911 is positioned at the end portion (section) of the flexible medical guidewire assembly 102. At least some of the plurality of terminal portions (409A, 409B, 409N) are spaced apart (angularly spaced apart) from each other along the outer surface of the jacket element 108.

Referring to the embodiment as depicted in FIG. 12A, the core terminal portion 106A extends (preferably) axially away from the end portion of the core element 106 from the end portion of the flexible medical guidewire assembly 102. The core terminal portion 106A forms (preferably) an elongated post (an electrically conductive post) having a keyed outer profile (such as a semicircular outer profile, etc.). The core terminal portion 106A forms (preferably) an axially-extending outer end flat side.

Referring to the embodiment as depicted in FIG. 12B, the electrical-connector assembly 810 is configured to interface (electrically interface) with the plurality of terminal portions (409A, 409B, 409N, 106A) as depicted in FIG. 12A. The push button 820 is mounted to the electrical-connector assembly 810, and is configured to selectively electrically connect the interior electrical components of the electrical-connector assembly 810 to the plurality of terminal portions (409A, 409B, 409N, 106A). The electrical-connector assembly 810 forms (provides) a connector channel 824 (preferably a keyed connector channel). The connector channel 824 (the keyed connector channel) is configured (formed or keyed) to receive a keyed (correspondingly keyed) terminal portion (preferably, the proximal end) of the flexible medical guidewire assembly 102, such as the core terminal portion 106A as depicted in FIG. 12A, etc. A leaf spring 836 is positioned on an inner surface (of the electrical-connector assembly 810) facing into the connector channel 824 (the keyed connector channel). The leaf spring 836 is configured to bias the plurality of terminal portions (409A, 409B, 409N, 106A) of FIG. 12A toward a relatively secure electrical connection (and mechanical connection) between the plurality of terminal portions (409A, 409B, 106A) and corresponding electrical contacts provided by the electrical-connector assembly 810. The channel 824 (the keyed connector channel) includes (preferably) a keyed mating groove 838 (a keyed slot) configured to mate (slidably receive) the core terminal portion 106A of FIG. 12A. The electrical-connector assembly 810 (with assistance from the leaf spring 836) is configured to permit axially sliding interface between the plurality of terminal portions (409A, 409B, 106A) and the electrical-connector assembly 810.

Referring to the embodiment as depicted in FIG. 12C, the electrical-connector assembly 810 is configured to provide a keyed over-the-wire connection. This arrangement provides (facilitates) keyed alignment between the plurality of terminal portions (409A, 409B, 409N, 106A) (also called the proximal electrode terminals) with spaced-apart spacing between the terminal portions. This arrangement mitigates the risk of incorrect electrical connections. The connector terminals 840 (of the electrical-connector assembly 810) are located in the interior of the connector channel 824 (the keyed connector channel), and are configured to electrically connect with the plurality of terminal portions (409A, 409B, 409N, 106A) once the end portion of the flexible medical guidewire assembly 102 is inserted into the connector channel 824 of the electrical-connector assembly 810.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the invention which does not materially modify the invention. Similarly, unless specifically made clear from its context, numerical values should be construed to include certain tolerances that the person skilled in the art would recognize as having negligible importance as they do not materially change the operability of the invention. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options would be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the invention. The foregoing has outlined the non-limiting embodiments (examples). The description is made for particular non-limiting embodiments (examples). It is understood that the non-limiting embodiments are merely illustrative as examples.

1-74. (canceled)

75. An apparatus, comprising:
a flexible medical guidewire assembly configured to be inserted into a confined space defined by a living body; and
a sensor assembly being securely supported by the flexible medical guidewire assembly in such a way that the sensor assembly and the flexible medical guidewire assembly are movable along the confined space defined by the living body once the flexible medical guidewire assembly is inserted into, and moved along, the confined space defined by the living body.

76. The apparatus of claim 75, wherein:
the flexible medical guidewire assembly includes a core element being electrically conductive; and
the flexible medical guidewire assembly also includes at least one electrical wire extending along a length of the flexible medical guidewire assembly; and
the sensor assembly is electrically connected, via said at least one electrical wire, to a proximal terminal portion of the flexible medical guidewire assembly; and
said sensor assembly and said at least one electrical wire are electrically isolated from the core element.

77. The apparatus of claim 75, wherein:
the sensor assembly includes at least one electrode.

78. The apparatus of claim 75, further comprising:
a radio frequency emitter supported by the flexible medical guidewire assembly; and
the radio frequency emitter is configured to cauterize tissue.

79. The apparatus of claim 78, wherein:
the flexible medical guidewire assembly includes a tip portion; and
the radio frequency emitter is mounted to the tip portion.

80. The apparatus of claim 79, further comprising:
a sharp mounted to the tip portion, the sharp configured to cut tissue.

81. The apparatus of claim 75, wherein:
the flexible medical guidewire assembly includes:
a core element; and
a jacket element surrounding the core element; and
the core element and the jacket element extend along an elongated length of the flexible medical guidewire assembly.

82. The apparatus of claim 81, wherein:
the flexible medical guidewire assembly includes Society of Automotive Engineering Type stainless steel.

83. The apparatus of claim 81, wherein:
the core element is electrically conductive.

84. The apparatus of claim 81, wherein:
the core element includes a hollow tube configured to receive and house an electrical wire.

85. The apparatus of claim 75, wherein:
the sensor assembly includes:
a magnetic sensor device or an electrical sensor device.

86. The apparatus of claim 75, wherein:
the sensor assembly includes:
an electrical sensor device configured to transmit an electrical signal.

87. The apparatus of claim 75, wherein:
the flexible medical guidewire assembly includes an electrical wire extending along a length of the flexible medical guidewire assembly; and
the electrical wire is electrically connected to the sensor assembly; and
the electrical wire extends from the sensor assembly toward a terminal end of the flexible medical guidewire assembly, and terminates at a terminal contact positioned at the terminal end of the flexible medical guidewire assembly.

88. The apparatus of claim 75, further comprising:
a sensor-interface system configured to interface with the sensor assembly; and
the sensor-interface system also configured to exchange a signal with the sensor assembly;
a signal measurement system; and
wherein the sensor-interface system also configured to be electrically connectable to the signal measurement system.

89. The apparatus of claim 75, wherein:
the flexible medical guidewire assembly comprises a distal electrode; and
the distal electrode comprises a radio frequency emitter, in which the radio frequency emitter is configured to provide energy of an amount to puncture adjacently positioned tissue of the living body positioned adjacent to the distal electrode, once the flexible medical guidewire assembly is inserted into the confined space defined by the living body, and once the distal electrode is activated; and
the sensor assembly comprises a plurality of electrical sensor devices being spaced apart from each other, and being fixedly positioned along a longitudinal length of the flexible medical guidewire assembly; and
a plurality of terminal portions are positioned as a proximal end of the flexible medical guidewire assembly; and
the plurality of terminal portions are electrically coupled to respective one of the plurality of electrical sensor devices; and
the plurality of terminal portions are configured to be selectively electrically connectable to, and removable from, an electrical-connector assembly.

90. A method of utilizing a flexible medical guidewire assembly, comprising:
providing the flexible medical guidewire assembly configured to be inserted into a confined space defined by a living body, in which there is a sensor assembly securely supported by the flexible medical guidewire assembly; and
inserting, at least in part, the sensor assembly and the flexible medical guidewire assembly into the confined space defined by the living body; and
moving, at least in part, the sensor assembly and the flexible medical guidewire assembly along the confined space defined by the living body once the flexible medical guidewire assembly is inserted into the confined space defined by the living body.

91. The method of claim 90, wherein:
the flexible medical guidewire assembly includes a core element being electrically conductive; and
the flexible medical guidewire assembly also includes at least one electrical wire extending along a length of the flexible medical guidewire assembly; and
the sensor assembly is electrically connected, via said at least one electrical wire, to a proximal terminal portion of the flexible medical guidewire assembly; and
said sensor assembly and said at least one electrical wire are electrically isolated from the core element.

92. The method of claim 90, wherein:
the sensor assembly includes at least one electrode.

93. The method of claim 90, wherein:
the sensor assembly includes a plurality of electrodes.

94. The method of claim 90, wherein:
a radio frequency emitter is supported by the flexible medical guidewire assembly; and
the radio frequency emitter is configured to cauterize tissue.
